(19) United States
(12) Patent Application Publication
(10) Pub. No.: US 2024/0212857 A1
(43) Pub. Date: Jun. 27, 2024

(54) DEEP NEURAL NETWORKS FOR PREDICTING POST-OPERATIVE OUTCOMES

(71) Applicant: CEDARS-SINAI MEDICAL CENTER, Los Angeles, CA (US)

(72) Inventors: David Ouyang, Los Angeles, CA (US); John Theurer, Los Angeles, CA (US)

(73) Assignee: CEDARS-SINAI MEDICAL CENTER, Los Angeles, CA (US)

(21) Appl. No.: 18/556,613

(22) PCT Filed: Apr. 21, 2022
(86) PCT No.: PCT/US22/25822

§ 371 Date: Oct. 20, 2023

Related U.S. Application Data
(60) Provisional application No. 63/177,489, filed on Apr. 21, 2021.
(60) Provisional application No. 63/197,023, filed on Jun. 4, 2021.

(51) Int. Cl. G16H 50/30; A61B 5/318; G16H 50/20

(52) U.S. Cl. CPC G16H 50/30; A61B 5/318; G16H 50/20

(57) ABSTRACT

Disclosed herein are systems and methods for evaluating perioperative risk based on electrocardiogram (ECG) signals. In one example, perioperative risk for a patient is predicted via a convolutional neural network model comprising at least one atrous layer and using pre-operative ECG data as input. Further, in one example, responsive to determining the perioperative risk metric below a threshold risk, selecting a patient for a surgical procedure and performing the surgery on the patient.

### CROSS REFERENCE TO RELATED APPLICATIONS

This application claims priority to U.S. Provisional Application No. 63/177,489, filed Apr. 21, 2021 titled DEEP NEURAL NETWORKS FOR PREDICTING POST-OPERATIVE OUTCOMES, and U.S. Provisional Application No. 63/197,023, filed Jun. 4, 2021, titled DEEP NEURAL NETWORKS FOR PREDICTING POST-OPERATIVE OUTCOMES, the contents of all of which are incorporated herein by reference.

### FIELD

The present invention relates to predicting post-operative outcomes using pre-operative electrocardiograms (ECGs).

### BACKGROUND

The following description includes information that may be useful in understanding the present invention. It is not an admission that any of the information provided herein is prior art or relevant to the presently claimed invention, or that any publication specifically or implicitly referenced is prior art.

Perioperative cardiovascular risk evaluation is an integral part of clinical decision-making performed prior to undertaking a surgical intervention. In order to asses perioperative risk, pre-operative electrocardiograms (ECGs) are obtained. Current risk stratification algorithms have variable accuracy and are time-consume consuming. Accordingly, there is a need for more accurate, reliable, and faster post-operative risk evaluation.

### SUMMARY

Patients undergoing major surgery are at risk for post-operative cardiovascular complications, such as myocardial infarction, heart block, arrhythmias, cardiac arrest, and in some cases, even death. To balance surgical risk with the potential benefits of intervention, perioperative risk evaluation aims to identify patients at increased risk of complications. An example approach for evaluating risk of post-operative cardiac complications is provided by Lee et al (Lee T. H. et al. Derivation and prospective validation of a simple index for prediction of cardiac risk of major noncardiac surgery. Circulation. 1999; 100: 1043-1049). Therein, a revised cardiac risk index (RCRI) is calculated based on pre-operative risk factors including high-risk surgery, history of ischemic heart disease, history of congestive heart failure (CHF), serum creatinine greater than 2 mg/dL, cerebrovascular disease, and diabetes requiring insulin. However, the RCRI has a lower predictive accuracy. Other existing risk evaluation approaches integrate clinical features, diagnoses, functional status, and the clinical scenario to assist in risk stratification prior to surgery. However, these scoring systems have subjective elements, potentially overestimate risk in low-risk patients, and/or require tedious manual data entry of clinical information from many sources. Another disadvantage of existing risk evaluation approaches is small cohort sizes used in validation. Some approaches employ post-operative biomarker levels to further risk stratification for post-operative mortality; however, the dependence on post-procedural assaying limits the opportunity to pre-operative risk stratification. Further, current deep learning models for ECG evaluation predict long-term mortality. Such distant interpretations often lack the actionability and cannot be applied for perioperative risk stratification and clinical decision making for surgical purposes.

The inventors herein have identified the above-mentioned issues. Further, the inventors have recognized that an ECG of a patient may include one or more hidden risk markers that can be utilized to prognosticate post-operative outcomes including mortality and non-fatal major adverse cardiac events (MACE). Accordingly, the inventors herein have developed systems and methods to at least partially address some of the disadvantages discussed above and to improve prediction of post-operative outcomes based on ECG. In one example, a method for performing a perioperative risk assessment of a patient before a surgical procedure, the method comprising: receiving, at a processor coupled to an ECG sensor system, an electrocardiogram (ECG) waveform data acquired from the ECG sensor system; and determining, by the processor, one or more post-operative risk metrics based on a trained neural network model, the trained neural-network model receiving the ECG waveform as input and outputting, via a display communicatively coupled to the processor, the one or more post-operative risk metrics of the patient; wherein the trained neural network model is trained based on a set of pre-operative ECGs acquired within a threshold duration before corresponding surgical procedures; and wherein the trained neural network model includes at least one atrous convolutional layer performing an atrous convolutional operation on the ECG waveform data.

In this way, by utilizing a neural network model trained to classify perioperative risk using pre-operative ECG waveform data of a patient obtained, a more accurate and faster assessment of cardiac-related risk that a patient may encounter post-surgery may be obtained.

As an example, a neural network model may include a first atrous convolutional layer followed by a series of inverted residual layers. The neural network model may be trained with a training dataset comprising a plurality of ECG waveform data acquired from patients prior to surgery and labelled with corresponding post-operative outcome such as death and/or adverse cardiac outcomes, such as cardiac arrest, myocardial infarction, heart block, and pulmonary edema. The neural network model is trained to extract ECG-based features from ECG waveforms and classify perioperative risk using the ECG-based features. Further, the neural network model applies an atrous convolution to the input ECG waveform, which may be pre-processed (e.g., filtered, normalized, etc.). By applying atrous convolution instead of down sampling at a first layer of the neural network model, feature resolution of the input ECG waveform is not degraded. As a result, predictive accuracy of the neural network model is improved.

Further, the series of inverted residual layers gradually increase initial input channels (e.g., from 12 channels for a 12-lead ECG data to 320 channels as output from a last inverted residual layer). By using inverted residual layers, computational efficiency is increased while improving feature representation, which is consistently preserved starting from the first atrous convolutional layer.

As a result, the trained neural network model provides improved accuracy of perioperative risk prediction at a faster rate. Further, the trained neural network model outputs perioperative risk using ECG data from a single acquisition.

Additionally, in some examples, for each prediction, one or more relevant ECG features extracted by the trained neural network model may be identified via an interpretable model. In one example, the one or more relevant ECG features may have a relevance score greater than a threshold relevance score. Thus, the one or more relevant ECG features that contributed to the prediction may be identified and indicated to the user. For example, the one or more important ECG features may be indicated via highlights on an ECG waveform graph that was used as input. The indication of the one or more important ECG features may be displayed on a user interface (e.g., as an overlay over an input ECG waveform graph, the waveform portion of the ECG including the one or more important ECG features may be enlarged and depicted separately along with the corresponding prediction and/or the input ECG waveform, etc.) in addition to indicating the post-operative risk output from trained the neural network model. In some examples, additionally or alternatively, one or more leads based on which the one or more important ECG features were identified may be determined and indicated.

In this way, by identifying the important ECG features (extracted from a given ECG data input) contributing to the corresponding post-operative risk prediction, and indicating the important ECG features along with a risk score or post-operative risk prediction, user confidence in the output prediction is increased, and improves decision-making process for surgery.

The above advantages and other advantages and features of the present description will be readily apparent from the following Detailed Description when taken alone or in connection with the accompanying drawings. It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The accompanying drawings, which are incorporated in and constitute a part of this specification, exemplify the embodiments of the present invention and, together with the description, serve to explain and illustrate principles of the invention. The drawings are intended to illustrate major features of the exemplary embodiments in a diagrammatic manner. The drawings are not intended to depict every feature of actual embodiments nor relative dimensions of the depicted elements, and are not drawn to scale.

FIG. 1 is a schematic diagram illustrating an electrocardiogram (ECG) processing system for perioperative risk prediction.

FIG. 2 is a schematic diagram illustrating an architecture of a convolutional neural network model for perioperative risk prediction, which can be used in the ECG processing system of FIG. 1, according to an embodiment of the disclosure;

FIG. 3A is a schematic diagram illustrating an architecture of an inverted residual and max pooling module included in the convolutional neural network model of FIG. 2, according to an embodiment of the disclosure;

FIG. 3B is a schematic diagram of a bottleneck module included in the inverted residual and max pooling module of FIG. 3A, according to an embodiment of the disclosure;

FIG. 4A is a schematic diagram of an example inverted residual and max pooling module, according to an embodiment of the disclosure;

FIG. 4B is a schematic diagram of an example inverted residual and max pooling module, according to another embodiment of the disclosure;

FIG. 5 is a high-level flow chart showing an example method for performing perioperative risk prediction, according to an embodiment of the disclosure;

FIG. 6 is a high-level flow chart showing an example method for training a convolutional neural network model for perioperative risk prediction, according to an embodiment of the disclosure;

FIGS. 7A and 7B are graphs illustrating model performance with respect to different step size and dilation for an atrous layer of a convolutional neural network model for perioperative risk prediction during hyperparameter tuning, according to an embodiment of the disclosure;

FIG. 8 shows an example graph illustrating an example pre-operative and post-operative timeline for ECG waveform selection in a patient population used for generating training datasets of pre-operative ECG waveforms for training a neural network model, such as neural network model at FIG. 2 or 12;

FIG. 9 shows a block diagram illustrating an example cohort selection procedure, according to an embodiment of the disclosure;

FIGS. 10A and 10B shows graphs of receiver operating characteristic curves depicting performance of a convolutional neural network model for perioperative risk prediction for mortality and adverse cardiac events, according to an embodiment of the disclosure;

FIGS. 11A and 11B show interpretability analysis of a convolutional neural network model for perioperative risk prediction, according to an embodiment of the disclosure;

FIG. 12 shows an example neural network model for perioperative risk prediction, according to an embodiment of the disclosure; and

FIG. 13 shows an example graphical user interface with example post-operative risk estimates for mortality and MACE.

In the drawings, the same reference numbers and any acronyms identify elements or acts with the same or similar structure or functionality for ease of understanding and convenience. To easily identify the discussion of any particular element or act, the most significant digit or digits in a reference number refer to the Figure number in which that element is first introduced.

### DETAILED DESCRIPTION

Unless defined otherwise, technical and scientific terms used herein have the same meaning as commonly understood by one of ordinary skill in the art to which this invention belongs. Szycher's Dictionary of Medical Devices CRC Press, 1995, may provide useful guidance to many of the terms and phrases used herein. One skilled in the art will recognize many methods and materials similar or equivalent to those described herein, which could be used in the practice of the present invention. Indeed, the present invention is in no way limited to the methods and materials specifically described.

In some embodiments, properties such as dimensions, shapes, relative positions, and so forth, used to describe and claim certain embodiments of the invention are to be understood as being modified by the term “about.”

Various examples of the invention will now be described. The following description provides specific details for a thorough understanding and enabling description of these examples. One skilled in the relevant art will understand, however, that the invention may be practiced without many of these details. Likewise, one skilled in the relevant art will also understand that the invention can include many other obvious features not described in detail herein. Additionally, some well-known structures or functions may not be shown or described in detail below, so as to avoid unnecessarily obscuring the relevant description.

The terminology used below is to be interpreted in its broadest reasonable manner, even though it is being used in conjunction with a detailed description of certain specific examples of the invention. Indeed, certain terms may even be emphasized below; however, any terminology intended to be interpreted in any restricted manner will be overtly and specifically defined as such in this Detailed Description section.

As used herein “perioperative risk assessment” or “perioperative risk evaluation” or “pre-operative risk assessment” refers to evaluation of risk of occurrence of a post-operative outcome after a surgical procedure, wherein the evaluation of risk is performed before the surgical procedure. In some implementations, “perioperative risk assessment” or “perioperative risk evaluation” or “pre-operative risk assessment” may be used to refer to evaluation of risk after a non-surgical procedure or treatment.

As used herein “pre-operative electrocardiogram (ECG)” refers to a ECG obtained at a time period before an elective cardiovascular or non-cardiovascular surgical procedure. In one example, pre-operative electrocardiogram may be obtained within 30 days before a surgical procedure. In some implementations, “pre-operative ECG” refers to a ECG obtained before a non-surgical procedure or treatment, and may be used to assess post-procedure risk of mortality and/or MACE using the trained neural networks described herein.

As used herein “post-operative event” or “post-operative outcome” refers to an event or outcome after a surgical procedure. The post-operative event or outcome may be mortality or a non-fatal major adverse cardiac event (MACE). The MACE may include but not limited to myocardial infarction (MI), arrhythmias, heart block, congestive heart failure (CHF), and pulmonary edema. In one example, the post-operative event or outcome may occur within a time-period after the surgical procedure. In one example, the time-period after the surgical procedure is 30 days after completion of the surgical procedure.

As used herein “post-operative risk metrics” refer to one or more metrics associated with a post-operative risk. The one or more metrics may include one or more of a probability of occurrence of one or more post-operative outcomes (e.g., MACE, mortality), a percentile at which the patient is at for each of the one or more post-operative outcome, an associated percentage corresponding to the percentile for each of the one or more post-operative outcomes, a degree of post-operative risk (e.g., high, medium, low, etc.) for each of the one or more post-operative outcomes, and a classification of post-operative risk (e.g., binary classification (e.g., for a given post-operative outcome, a corresponding post-operative risk above a threshold level exists or not), multi-level classification (e.g., level 1 associated with low risk, level 2 associated with moderately low risk, and so on))

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations may be depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

### Overview

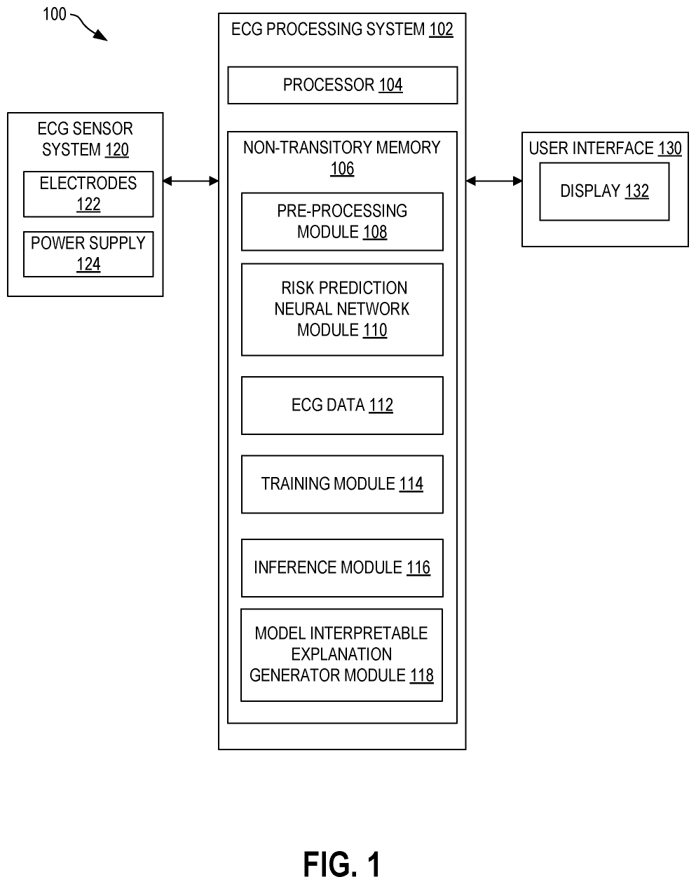

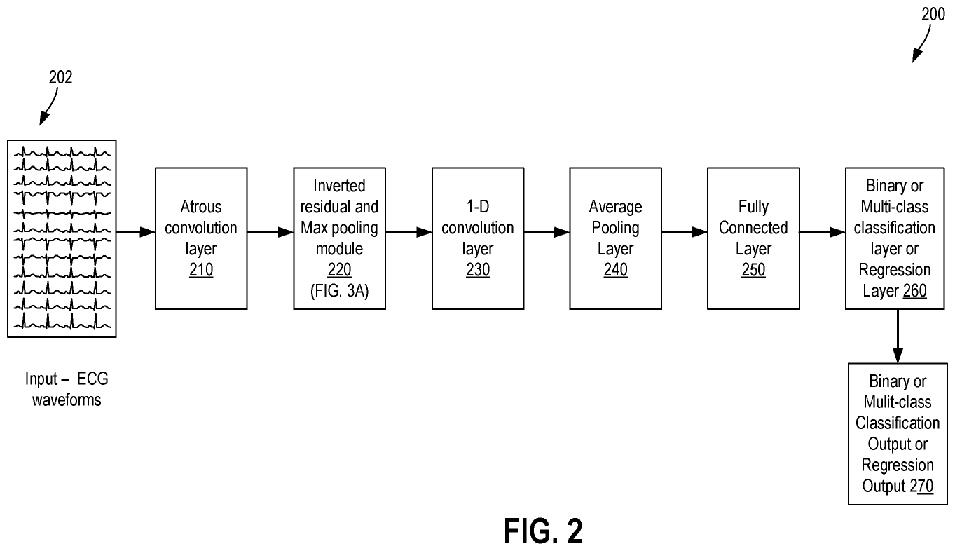

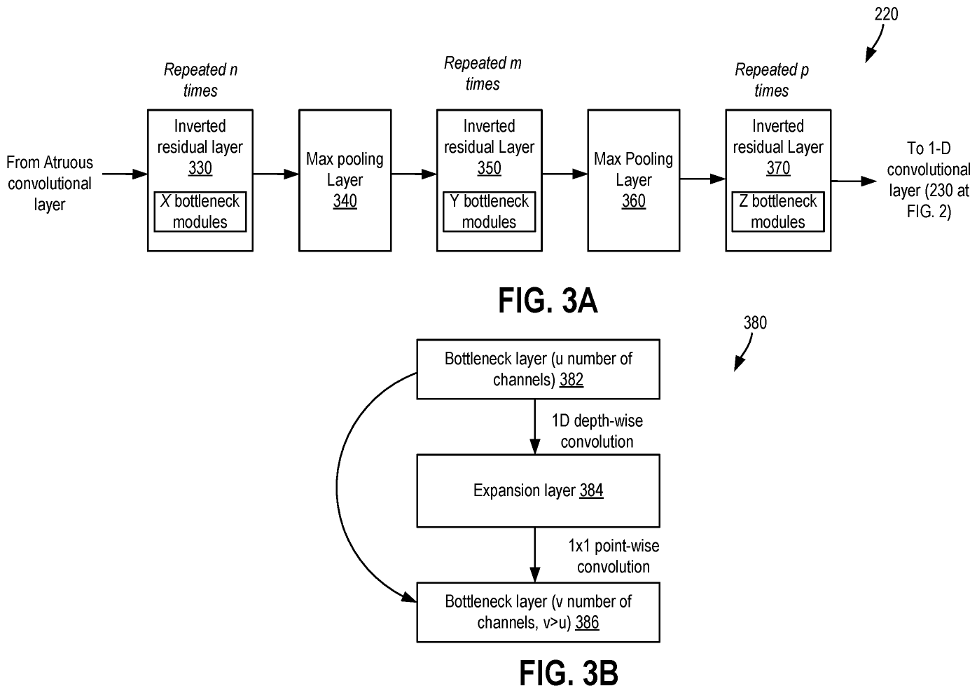

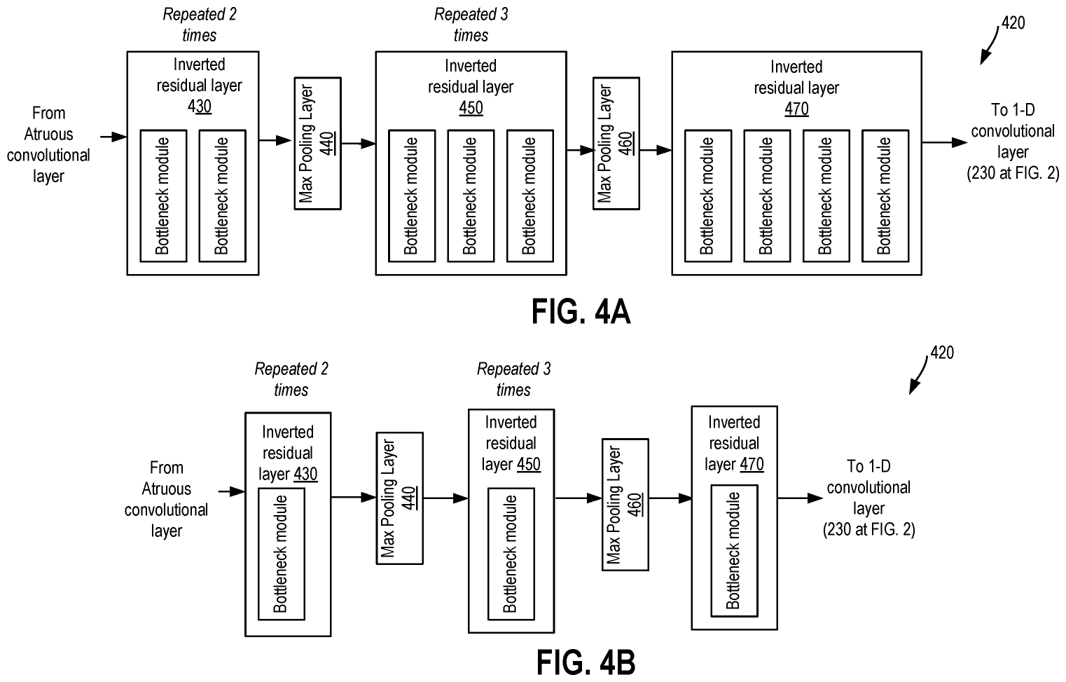

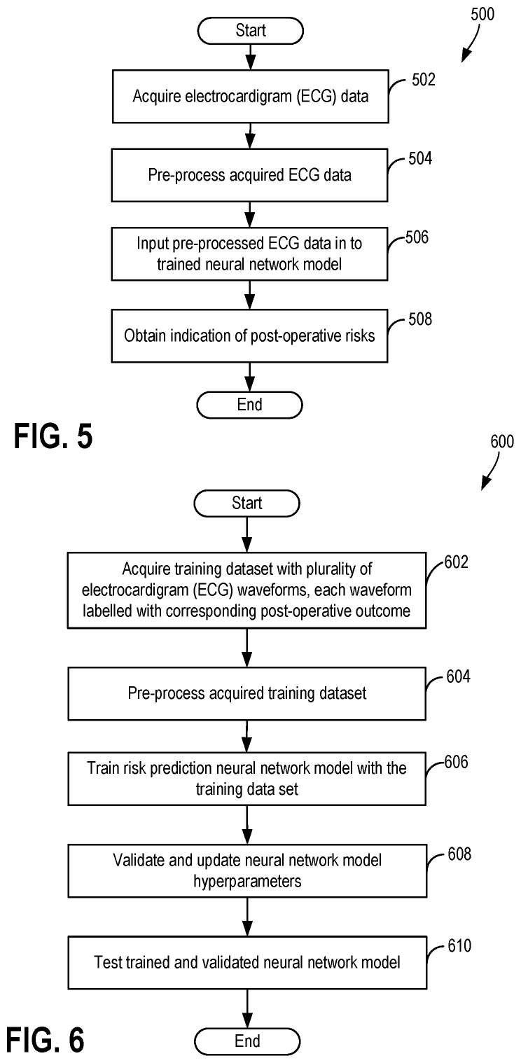

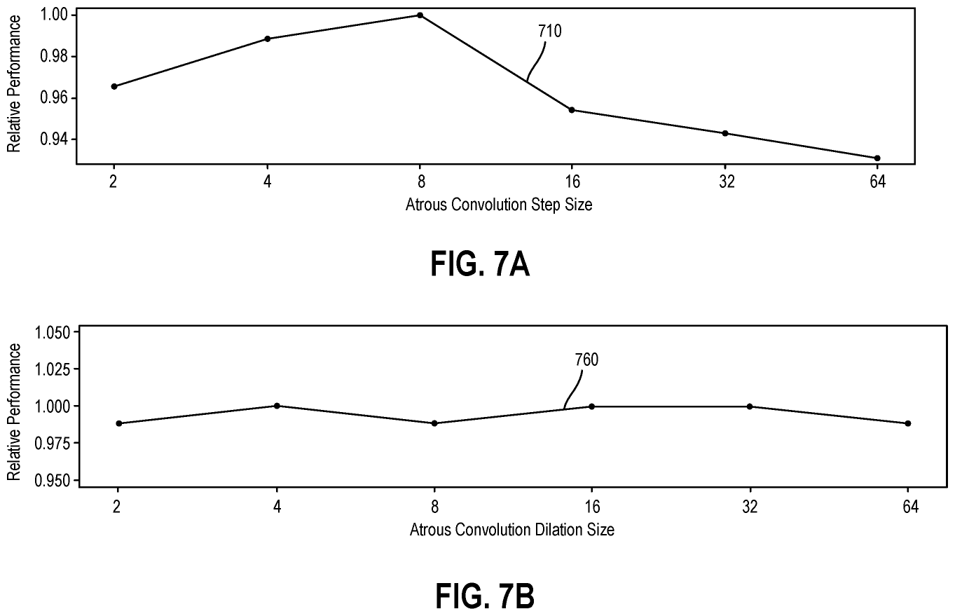

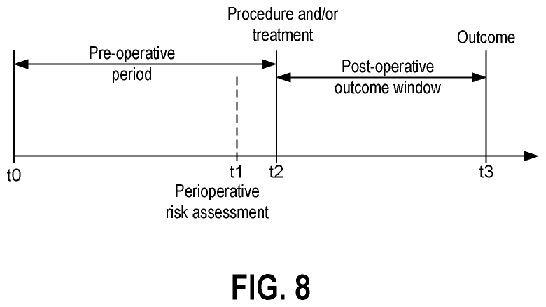

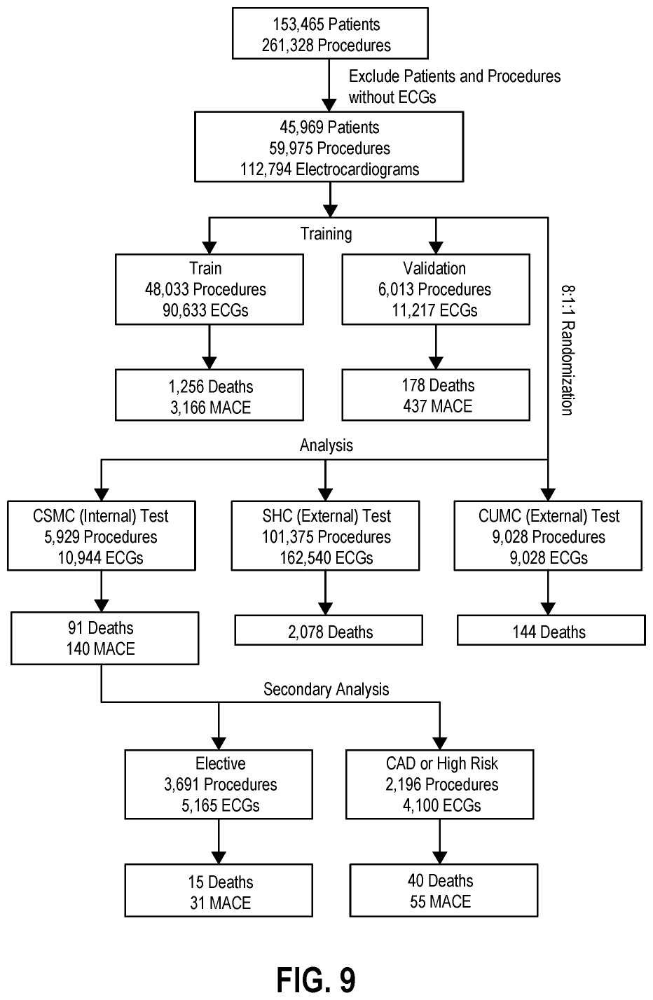

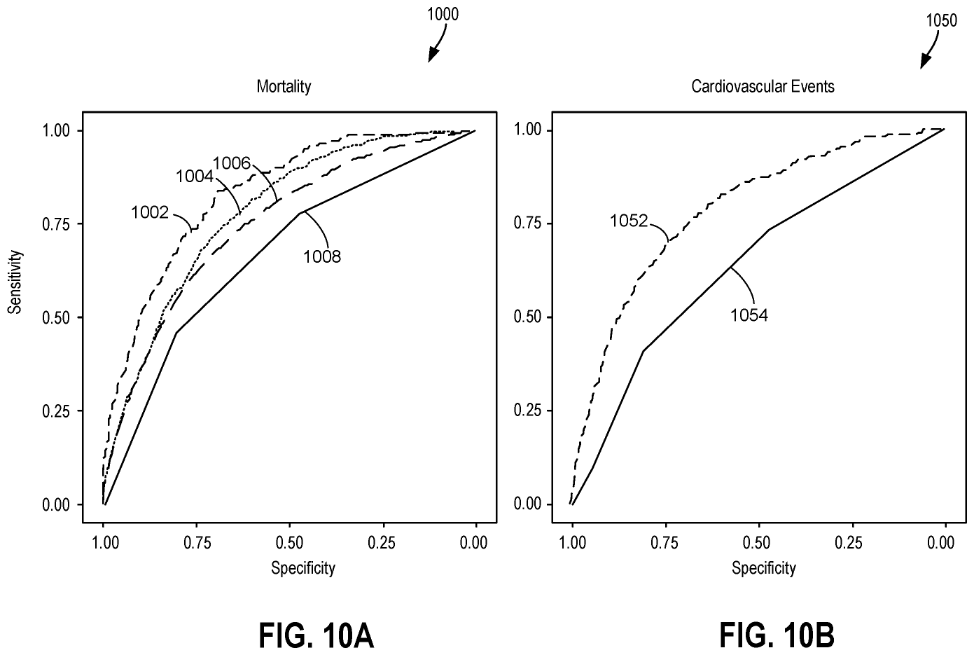

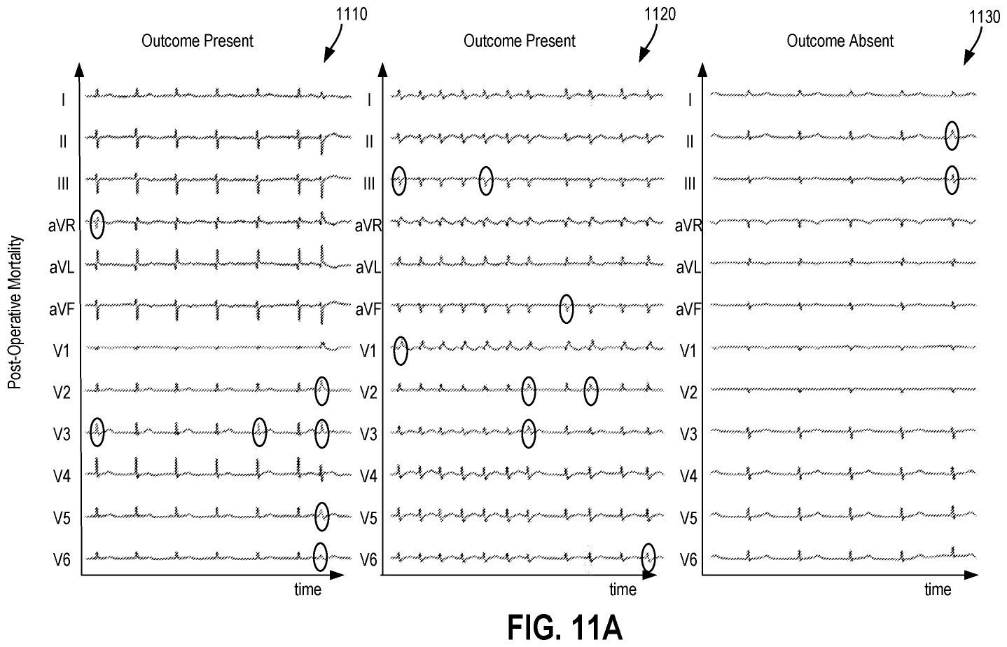

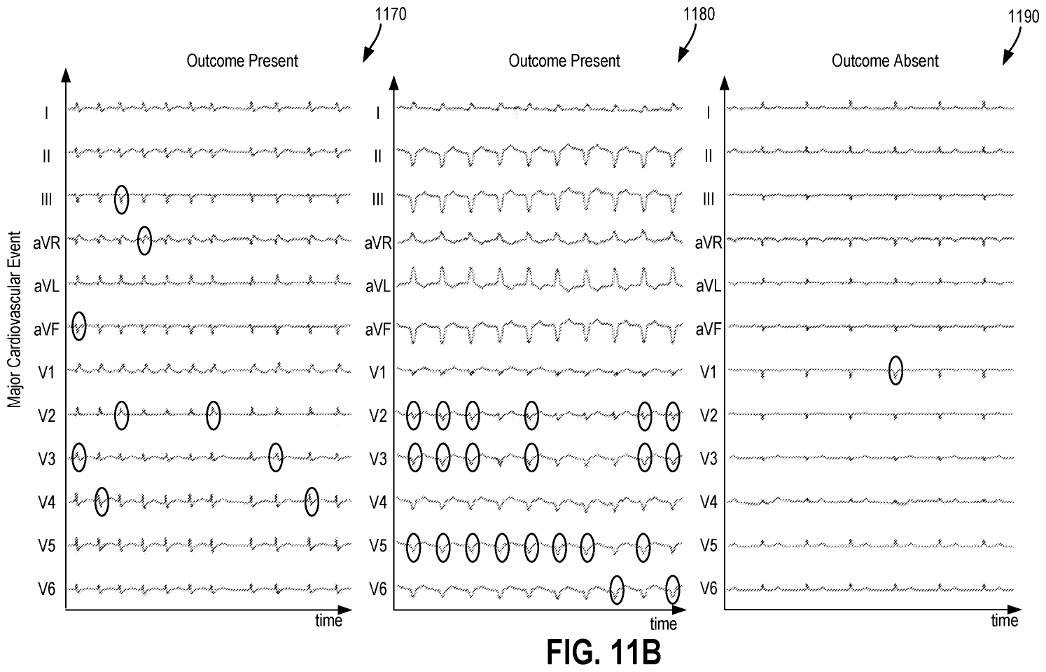

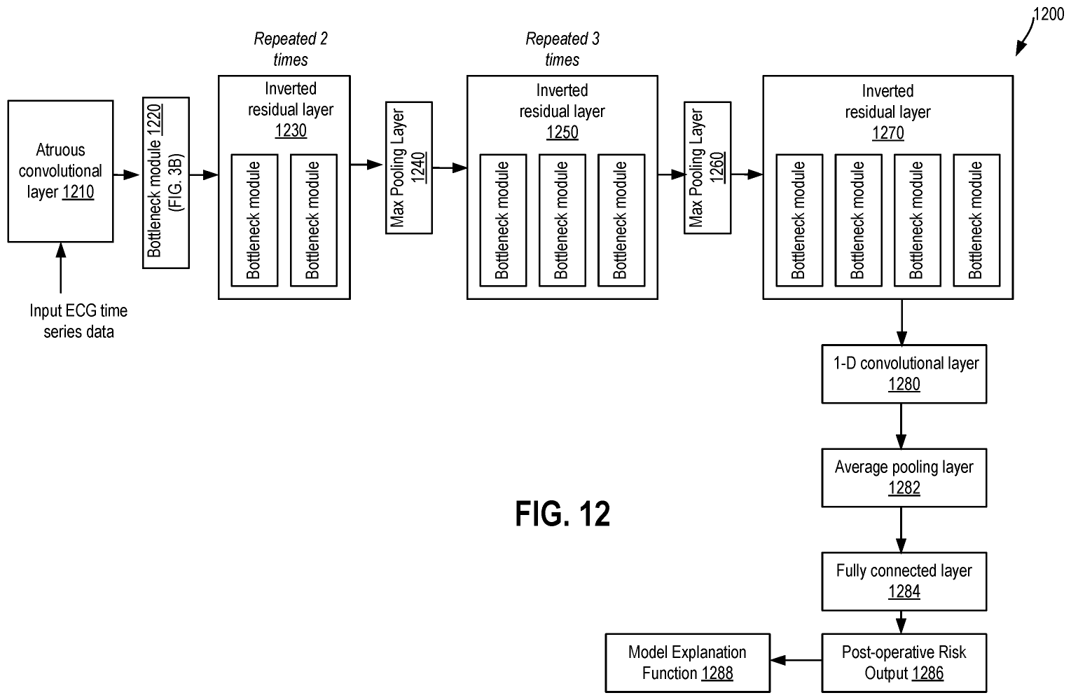

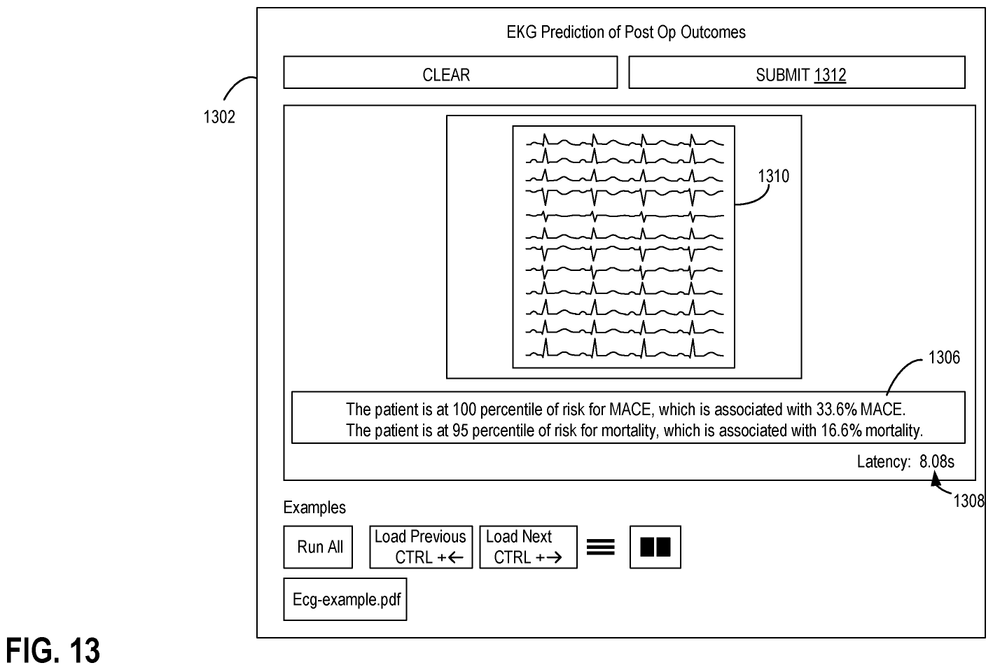

Disclosed herein are systems and methods for perioperative risk evaluation by implementing a convolutional neural network model, such as the convolutional neural network model for perioperative risk evaluation at FIG. 2. The convolutional neural network model is alternatively referred to herein as the neural network model. The convolutional neural network model may process an ECG of a patient acquired within a threshold time period before a surgical procedure, and generate a perioperative risk metric that predicts one or more post-operative outcomes based on the ECG. The convolutional neural network model may be implemented in an ECG processing system, such as the ECG processing system of FIG. 1. The convolutional neural network model comprising at least one atrous layer followed by an inverted residual and max pooling module that includes a series of inverted residual layers. A high-level architecture of the inverted residual and max pooling module is shown at FIG. 3A, and a repeating bottleneck module of an inverted residual layer of the series of inverted residual layers is shown at FIG. 3B. Further FIGS. 4A and 4B show various embodiments of the inverted residual and max pooling module. While the atrous convolution layer preserves the resolution of the input ECG waveform, the inverted residual layers increase computational efficiency. As a result, predictive accuracy as well as processing speed of the neural network model is improved. An example method for training a convolutional neural network model, such as the neural network model at FIG. 2, for perioperative risk prediction is shown at FIG. 6 and an example method for obtaining a perioperative risk evaluation is shown at FIG. 5. Examples of hyperparameter tuning during training of the convolutional neural network model are shown at FIGS. 7A and 7B. Further, an example cohort selection procedure for training, validating, and testing the convolutional neural network model is shown at FIG. 9. A pre-operative time period when the ECG waveform data is obtained before the procedure or treatment, and a post-operative window when post-operative adverse outcomes are considered is shown at FIG. 8. An example receiver operating characteristic graphs showing performance of the convolutional neural network model are shown at FIGS. 10A and 10B. An example interpretability analysis output identifying and visually showing relevant features in an example ECG used for model decision making is shown at FIGS. 11A and 11B. Another example neural network model is shown at FIG. 12. Further, an example output on a web interface for the neural network model is depicted at FIG. 13. The convolutional neural network model is trained with ECG waveform data acquired from a large cohort of patients undergoing elective procedure over a decade, and labeled with observed post-operative outcomes, the post-operative outcomes including one or more of mortality and adverse cardiac event. Thus, the convolutional neural network model is trained to predict one or more post-operative outcomes including one or more of mortality and adverse cardiac events.

A technical advantage of the convolutional network models described herein for perioperative risk prediction is a relative few number of parameters and high computational efficiency compared to existing neural network architectures for interpreting ECG with improvement in predictive performance. Being up to one hundred times smaller than existing models, the convolutional neural network models described herein can be deployed without graphical processing units, which are less common in clinical settings. Further, the convolutional neural network models described herein require less time to run inference (e.g., than 1 second). Another technical advantage is improved surgical risk prediction as the convolutional neural network models are trained with ECG datasets from the large cohort of patients undergoing elective procedure across a decade and labelled with multiple granular perioperative outcomes, and the training data is labelled with hospitalization outcomes instead of long-term outcomes. As a result, perioperative risk stratification provided by the convolutional neural network model enables clinical prognostication of surgical risk (which is not possible with existing deep learning algorithm that simply analyze ECG) which allows the clinicians to take necessary actions prior to surgery. Another technical advantage is that the trained convolutional neural network model predicts perioperative risk with increased accuracy and efficiency using a single pre-operative ECG acquisition prior to a procedure. As a result, decision making process is greatly expedited, which in turn improves patient outcome. Yet another technical advantage is by applying an interpretable model for each prediction, and identifying the important ECG features contributing to each prediction, clinician confidence in the trained neural network model and the prediction output by the neural network model is increased, which further expedites the decision making process.

In this way, the systems and methods described herein provide an important step towards streamlining perioperative risk evaluation. Recognizing that human experts have only limited intuition for prognosticating post-procedural outcomes and limitations of conventional risk calculators including tedious data entry and small cohort sizes used in validation, the inventors herein have developed convolutional neural network models that provide increased predictive accuracy and faster run times. As a result, the system and methods based on convolutional neural network models described herein provide significant improvement in post-operative risk stratification and enables improved patient outcome post-operatively.

In various embodiments, the methods and systems described herein may be utilized for non-surgical risk stratification. Accordingly, in one example, the neural network model may be trained to estimate post-procedure risk that includes evaluation of mortality and/or cardiovascular event risk after minor procedures. In this example, the training dataset may include ECG waveform data from patients who have undergone the minor procedures, where the ECG waveform data is acquired within a threshold period before the procedure and labelled with post-procedure outcomes for mortality and/or cardiovascular events. In another example, the neural network model may be trained to estimate post-treatment risk that includes evaluation of mortality and/or cardiovascular event risk after treatments, such as chemotherapy, radiation therapy, etc. In this example, the training dataset may include ECG waveform data from patients who have undergone the treatments (e.g., chemotherapy, radiation therapy, etc.), where the ECG waveform data is acquired within a corresponding threshold period before commencement of the treatment and labelled with post-procedure outcomes for mortality and/or cardiovascular events. In yet another example, the neural network model may be trained for non-surgical risk stratification, for example, to estimate life expectancy.

### Systems

Referring to FIG. 1, an electrocardiogram (ECG) system 100 is shown, in accordance with an exemplary embodiment. In some embodiments, as shown, the ECG system 100 comprises a ECG processing system 102 communicatively coupled to an ECG sensor system 120 including two or more ECG leads 122. The ECG sensor system 120 may further include a power supply 124 to provide electrical power to the two or more ECG electrodes 122. In some examples, the ECG sensor system 120 may include two or more lead wires (not shown) for transmitting ECG electrical signal from a given lead to an integrated processing unit integrated with the ECG sensor system 120. Further, the ECG electrical signals may be processed by the integrated processing unit and subsequently transmitted (via a wired and/or a wireless connection) to the ECG processing system 102 for perioperative risk evaluation. In some examples, the ECG electrical signal may be wirelessly transmitted to the integrated processing unit and/or the ECG processing system 102 communicatively coupled to the ECG sensor system 100. In one example, the ECG sensor system 120 may be configured to generate 12-lead ECG data based on 12 ECG leads representing a patient's heart electrical activity acquired from 10 or more electrodes. In another example, the ECG sensor system 120 may be configured to generate 12-lead ECG data from fewer than 10 ECG electrodes. While in some examples, 12-lead ECG data is used for perioperative risk prediction, it will be appreciated that fewer lead ECG data from any number of electrodes (that is, any number less than 12) may be used for perioperative risk evaluation using a neural network model as further discussed below.

In one example, the ECG system 100 may be a clinical grade ECG system. In another example, the ECG system may be configured as a wearable device. The wearable may be a smart watch, smart ankle bracelet, smart glasses, smart ring, patch, band, digital stethoscope, or other device that suitably could be retained on a patient and give access to the patient's skin to various sensors on the wearable. In some examples, the wearable may include adhesives and stick onto a patient's skin on the neck, chest, arm, leg, torso, back or other suitable locations. In some other examples, the ECG system may be configured as a Holter monitor, multi-channel ECG (MECG), or implantable loop recorder.

In some embodiments, the ECG processing system 102 is disposed at a remote device (e.g., edge device, server, etc.) communicably coupled to the ECG sensor system 120 via wired and/or wireless connections. In some embodiments, the ECG processing system 102 is disposed at a separate device (e.g., a clinical workstation comprising a computer) which can receive ECG signals from the ECG sensor system 120 or from a storage device which stores the ECG data acquired by the ECG sensor system 120. The ECG processing system 102 may comprise at least one processor 104, and a user interface 130 which may include a user input device (not shown), and a display device 132. User input device may comprise one or more of a touchscreen, a keyboard, a mouse, a trackpad, a motion sensing camera, or other device configured to enable a user to interact with and manipulate data within the ECG processing system 102.

The ECG processing system 102 comprises at least one processor 104 configured to execute machine readable instructions stored in non-transitory memory 106. The processor 104 may be single core or multi-core, and the programs executed thereon may be configured for parallel or distributed processing. In some embodiments, the processor 104 may optionally include individual components that are distributed throughout two or more devices, which may be remotely located and/or configured for coordinated processing. In some embodiments, one or more aspects of the processor 104 may be virtualized and executed by remotely-accessible networked computing devices configured in a cloud computing configuration. According to other embodiments, the processor 104 may include other electronic components capable of carrying out processing functions, such as a digital signal processor, a field-programmable gate array (FPGA), or a graphic board. According to other embodiments, the processor 104 may include multiple electronic components capable of carrying out processing functions. For example, the processor 104 may include two or more electronic components selected from a list of electronic components including: a central processor, a digital signal processor, a field-programmable gate array, and a graphic board.

In still further embodiments the processor 104 may be configured as a graphical processing unit (GPU) including parallel computing architecture and parallel processing capabilities. However, it will be appreciated that a trained neural network model as described herein for perioperative risk evaluation may be implemented in a processor that does not have GPU processing capabilities. As a non-limiting example, the trained neural network model may be implemented as a decision support tool in a clinical setting. For example, the trained neural network model may be installed and run on a computing device (e.g., standard-build clinical workstation comprising a computer) without graphical processing units (GPUs). Further, in some examples, a web application accessible via a computing device (e.g., a computer, a tablet, a mobile device, etc.) may be implemented to facilitate access by a user (e.g., a clinician). Accordingly, in one implementation, the computing device may include a web application, which represents machine executable instructions in the form of software, firmware, or a combination thereof. The components identified in the web application may be part of an operating system of the computing device or may be an application developed to run using the operating system. In some examples, the web application may mirror a mobile application implemented on a mobile device, e.g., providing the same or similar content as the mobile application.

In one example, the web application may include a graphical user interface and may, in some examples, assist a clinician in acquiring ECG sensor data for perioperative evaluation. For example, the web application may be used to initiate ECG sensor data acquisition from the ECG sensor system 120. Further, in one example, the web application may be configured to receive ECG sensor data from the ECG sensor system 120, pre-process the ECG sensor data (e.g., apply filtering, normalizing to improve signal quality and reduce noise), and transmit the pre-processed sensor data to the ECG processing system 102 for perioperative risk evaluation.

In some examples, through the graphical user interface, the web application may (1) display acquired ECG waveform, (2) display perioperative risk assessment output and/or (3) provide indications of one or more ECG features that contributed the perioperative risk output, among other output and/or indications based on the perioperative risk assessment output. An example graphical user interface is shown at FIG. 13.

Further, the trained neural network model described herein may intake ECG waveform image data and output post-operative risk estimates at a faster rate. As non-limiting examples, the inventors have demonstrated that the trained neural network model accessed on a standard clinical workstation (e.g., Windows 10 64-bit operating system, 3.8 GHz processor) was able to intake image data from 50 de novo ECGs and output post-operative risk estimates within 0.032±0.004 seconds per ECG for the local software installation and 0.041±0.006 seconds per ECG for the web application accessed by a mobile phone. In this way, the method for post-operative risk estimation using a perioperative ECG image data of a patient based on the trained neural network models described herein provides technical effects of improved speed and accuracy for post-operative risk estimates.

Non-transitory memory 106 may store a pre-processing module 108, a risk prediction neural network module 110, and ECG data 112. The risk prediction neural network module 110 may include one or more neural network models, each neural network model comprising a plurality convolutional layers. The risk prediction neural network module 110 may further include instructions for implementing the one or more neural network models to receive an ECG waveform data of a patient acquired from an ECG sensor system and output a corresponding post-operative risk classification (e.g., high, low, intermediate) for mortality and/or one or more of cardiovascular adverse conditions. For example, the risk prediction neural network module 110 may store instructions for implementing a neural network model, such as an exemplary deep neural network shown at FIG. 2 or FIG. 12. The risk prediction neural network module 110 may include trained and/or untrained neural networks and may further include various data, or metadata pertaining to the one or more neural networks stored therein. In one example, the risk prediction module 110 may include a first neural network model trained to evaluate and/or estimate a risk of post-operative mortality. Additionally or alternatively, the risk prediction module 110 may include a second neural network model trained to evaluate and/or estimate a risk of post-operative MACE.

Non-transitory memory 106 may further store training module 114, which comprises instructions for training one or more neural network models stored in the risk prediction neural network module 110. Training module 114 may include instructions that, when executed by processor 104, cause ECG processing system 100 to conduct one or more of the steps of method 600 (FIG. 6) for training one or more neural network models with corresponding training data sets, discussed in more detail below. In some embodiments, training module 114 includes instructions for implementing one or more gradient descent algorithms, applying one or more loss functions, and/or training routines, for use in adjusting parameters of one or more neural network models of the risk prediction neural network module 110.

Non-transitory memory 106 also stores an inference module 116 that comprises instructions for validating and testing new data with the trained neural network model. Non-transitory memory 106 further stores ECG data 112. ECG data 114 includes for example, ECG waveforms acquired by an ECG sensor system. In some embodiments, ECG data 112 may include a plurality of training sets, each comprising a plurality of ECG waveforms.

In some embodiments, the non-transitory memory 106 may include components disposed at two or more devices, which may be remotely located and/or configured for coordinated processing. In some embodiments, one or more aspects of the non-transitory memory 106 may include remotely-accessible networked storage devices configured in a cloud computing configuration.

Non-transitory memory 106 may further store a model interpretable explanation generator module 118. The model interpretable explanation generator module 118 may include an interpretable model configured to provide one or more visual indications for explaining each individual prediction of the neural network model for post-operative risk prediction generated during perioperative risk assessment. The interpretable model may be based on a model interpretability technique (e.g., Local Interpretable Model-Agnostic Explanations (LIME)) that explains how a model made a determination. For example, the model interpretability technique provides an indication of one or more features that contributed to the classification/regression model determining a classification label (e.g., post-operative risk estimate) for input data (e.g., ECG data). In one example, the indication may be one or more of a highlight of one or more individual ECG waveforms (e.g., PQRST wave) corresponding to an electrical signal recorded via the electrodes, a segment of a plurality of ECG waveforms including the one or more important ECG features, a list of ECG leads and/or list of changes (e.g., amplitude changes, width changes, onset changes) in each of the listed leads that contributed to the classification (that is, post-operative risk prediction) by the neural network model for perioperative risk prediction.

The interpretable model may be a local interpretable model. In some examples, a global interpretable model may be used. Thus, via the model interpretable explanation generator, one or more important ECG features having a weightage greater than a threshold weightage and contributing to post-operative risk prediction may be identified and indicated. For example, when the neural network model of the risk prediction module 110 is configured to predict a primary outcome of mortality risk, one or more important ECG features that contribute to mortality risk prediction may be identified via the interpretable model and an ECG waveform including one or more indications of the one or more important ECG features that contributed to the mortality risk prediction may be generated.

In one examples, each of the one or more important ECG features may have a weightage greater than a threshold weightage. In one example, the weightage is based on a similarity score obtained via perturbation of a corresponding ECG feature. For example, in order to identify relevant features in an ECG waveform (that is, in order to identify one or more important ECG features that contributed to a given perioperative risk prediction), a percentage of the ECG waveform is iteratively randomly perturbed, and changes that most impacted neural network model performance is identified.

In some examples, the feature selection of the one or more important ECG features may be based on any of a weightage, a lasso fit or a forward selection (wherein features are added one by one based on their improvements to a ridge regression fit of the neural network model outcome) or other feature selection methods may be used.

In one example, the interpretable model is based on a Local Interpretable Model-agnostic Explanations (LIME) technique for identifying one or more important ECG features that contribute to the outcome of the neural network model. The output of the interpretable model, that is the indications of the one or more important ECG features that contribute to perioperative risk evaluation may be displayed via the user interface along with the output of the neural network model. For example, via the LIME technique, ECG waveform features that have the most influence on the model's prediction decisions may be highlighted.

In one example, the interpretable model (e.g., LIME technique) is applied with respect to a trained neural network model, such as a neural network model at FIGS. 2-4B, and FIG. 12 described herein, trained to predict post-operative risk. As such, the interpretable model is used to explain a post-operative risk prediction by highlighting the ECG features that contributed to the post-operative risk prediction.

In another example, the interpretable model (e.g., LIME technique) is used during a training process to optimize various parameters for the neural network model (e.g., neural network model at FIGS. 2-4B, and FIG. 12), including training parameters, for perioperative risk prediction. As a non-limiting example, one or more of a number of convolutional layers, a number of bottleneck modules, and a number of ECG leads may be adjusted during training/model optimization based on the interpretable model.

In yet another example, the interpretable model (e.g., LIME technique) is used during training of the neural network model, and on a trained neural network model to explain the predictions.

While the above examples describe using the LIME technique for interpreting the neural network model output, other interpretability techniques, such as Shapely Additive Explanations, may be used and are within the scope of the disclosure.

Display 132 may include one or more display devices utilizing virtually any type of technology. In some embodiments, display device 132 may comprise a computer monitor, and may display unprocessed and processed ECG waveforms. Display device 132 may be combined with processor 104, non-transitory memory 106, and/or user input device in a shared enclosure, or may be peripheral display devices and may comprise a monitor, touchscreen, projector, or other display device known in the art, which may enable a user to view ECG waveforms, and/or interact with various data stored in non-transitory memory 106.

It should be understood that ECG processing system 102 shown in FIG. 1 is for illustration, not for limitation. Another appropriate ECG processing system may include more, fewer, or different components. The ECG processing system 102 may be used to train and deploy a neural network model, such as an example neural network model discussed below at FIG. 2.

### Neural Network Architecture.

Turning to FIG. 2, it shows a high-level block diagram of a neural network model 200 that may be implemented for evaluating a post-operative risk of a patient for mortality and/or cardiovascular events based on ECG data acquired before the patient undergoes a surgical procedure. The neural network model 200 may be implemented based on instructions stored in non-transitory memory, such as non-transitory memory 106 of the ECG processing system 102 at FIG. 1. The neural network model 200 may receive as input, ECG waveform data of a patient, and output one or more post-operative risk metrics.

In one example, the ECG waveform data is time-series data. Further, the ECG waveform data may be pre-processed prior to passing through the neural network model 200. Pre-processing ECG waveform data may include filtering and normalization, for example. In some examples, filtering may be performed to remove very low frequency signals. Further, in some examples, normalization may be performed based on mean and SD of the ECG waveform data. In some other examples, normalization may be performed to move the ECG signals to a desired amplitude range (e.g., all positive valves, −1 to +1 etc.). In some examples, The ECG waveform data may be pre-processed to generate a desired ECG segment in a desired time window (e.g., time duration in a range between 10 seconds and 2 seconds). Thus, in one example, the ECG waveform data input into the neural network model for post-operative risk prediction may be a 10 second time series ECG waveform data. In some examples, a 2. 5 second ECG waveform data may be used. In particular, the neural network models discussed herein for post-operative risk prediction can be used with 2.5 s clips of ECG waveform data. In various examples, the ECG waveform data may be 10, 9.5, 9, 8.5, 8, 7.5, 7, 6.5, 6, 5.5, 5, 4.5, 4, 3.5, 3, 2.5, or 2 seconds. In any case, pre-processing of the input ECG signals may be based on the pre-processing operations performed during training the neural network model.

In one example, the neural network model 200 may be trained using a first training dataset comprising a plurality of first perioperative ECG waveforms, each of the first perioperative ECG waveforms labelled with an observed primary outcome during a time period post-surgery (e.g. mortality due to cardiovascular complications or no mortality during the post-surgery time period). In one example, the time period post-surgery corresponds to a period of hospitalization after the surgery or non-surgical procedure or treatment. In another example, the time period post-surgery is 30 days post-surgery (which may optionally include the day of surgery or non-surgical procedure or treatment). The time period post-surgery may be fewer than 30 days or greater than 30 days. As non-limiting examples, the time period post-surgery (or post non-surgical procedure or post treatment) for which the post-operative outcome is generated during pre-operative assessment may be in a range from 30 days to 90 days. In some example, the time-period post-surgery for which post-operative outcome is generated may be in a range from 90 days to 120 days. Accordingly, the post-operative risk metric may be an indication of post-operative mortality. For example, the indication may be a degree of risk (e.g., high, intermediate, low), a percentage of risk, a probability of occurrence, a patient percentile (that is, a percentile at which the patient is at risk), or a combination thereof of post-operative mortality. It will be appreciated that any binary or multi-level classification of risk of post-operative mortality may be determined using the neural network model 200, and is within the scope of the disclosure. An example post-operative mortality outcome indicated on a graphical user interface is shown at FIG. 13.

Turning to FIG. 13, it shows am example graphical user interface (GUI) 1302 on a display portion of a display of a clinical workstation that may be used for risk estimates. The GUI 1302 comprises a portion 1304 displaying an ECG waveform 1310. Further, the risk estimate for the ECG waveform, as determined by a neural network model, such as neural network model 200, is provided at results field 1306. In some examples, additionally, an explanation output from an explanation model, such as LIME, is additionally displayed or may be navigated via a button. Example explanation outputs that highlight the ECG features that contributed to a given risk estimate (e.g., mortality or MACE) may be generated via the LIME model, and indicated to the clinician via the user interface. Example explanation outputs on ECG time-series waveform data is shown at FIGS. 11A and 11B.

Accordingly, the GUI 1302 may be implemented for performing pre-operative assessment for risk of a post-operative mortality and/or cardiac adverse event outcome. A user may input an ECG waveform data 1310 (e.g., a 10 second ECG time-series data, a 2.5 second ECG time series data, etc.) via the user interface 1302. In some examples, the user may input a plurality of ECGs for analysis by the trained neural network model for the same subject or different subjects. Upon uploading the ECG waveform data, the user may press the submit button 1312 for analysis. Responsive to receiving the ECG waveform data, the trained neural network may process the one or more ECGs and output one or more predictions or one or more risk score for one or more of a post-operative mortality outcome and post-operative cardiac adverse event outcome (MACE). The output may be displayed within the results field 1306. In some examples, as shown, in addition to the output, the input ECG waveform may be displayed. Further, as will be discussed below and shown at FIGS. 11A and 11B, one or more indications highlighting one or more important ECG features that are relevant (e.g., having a relevance score greater than a threshold) to the risk scores for mortality and/or adverse cardiac events may be displayed on the waveform 1310.

In another example, the neural network model 200 may be trained using a second training dataset comprising a plurality of second pre-operative ECG waveforms, each of the pre-operative ECG waveforms labelled with an observed secondary outcome during the perioperative time period post-surgery. As discussed above with respect to post-operative mortality, in one example, the time period post-surgery corresponds to a period of hospitalization after surgery. In another example, the time period post-surgery is 30 days post-surgery (which may optionally include the day of surgery). The time period post-surgery may be fewer than 30 days or greater than 30 days. As non-limiting examples, the time period post-surgery for which the post-operative outcome is generated may be in a range from 30 days to 240 days. The secondary outcomes may comprise adverse cardiac events including myocardial infarction, heart block, heart failure, and pulmonary edema. Accordingly, the post-operative risk metric may be an indication of post-operative adverse cardiac events. For example, the indication may be a degree of risk (e.g., high, intermediate, low), a percentage of risk, a probability of occurrence, a patient percentile (that is, a percentile at which the patient is at risk), or a combination thereof of post-operative adverse cardiac events or major adverse cardiac events. It will be appreciated that any binary or multi-level classification of risk of post-operative cardiac events or a regression output indicating risk of post-operative cardiac events may be determined using the neural network model 200, and are within the scope of the disclosure. An example post-operative MACE outcome indicated on a graphical user interface is shown at FIG. 13.

In some examples, the post-operative risk metric may be a regression output providing a probability of risk (e.g., risk of post-operative mortality or risk of post-operative cardiac adverse event) for a given input ECG waveform.

The neural network model 200 includes a first atrous convolutional layer 210, wherein an atrous convolutional operation is applied to the input ECG waveform (that is, pre-processed input ECG waveform). The atrous convolutional operation applies a kernel with a desired dilation rate, desired size, and desired stride. The size and dilation rate of the kernel specifies a field of view for the atrous convolution operation. In one example, during training, the atrous convolution's dilation and step size is grid-searched by hyperparameter tuning for optimal area under curve (AUC) with all other hyperparameters held constant. Example hyperparameter sweep for dilation rate and step size (that is, stride) for atrous convolution is shown at FIGS. 7A and 7B. Atrous convolution is performed on the input ECG waveform in order to maintain higher resolution of ECG features, which are otherwise lost when downsampled by regular filters without atrous convolution. By implementing the first atrous convolutional layer 210, accuracy of risk prediction is improved. Previous neural network models on ECGs required input of downsampled ECGs given limitations of model size and computational power. By using the first atrous convolutional layer, the neural network model 200 allows for input of the full-resolution. Further, the atrous convolution is performed on each channel of the input ECG waveform. For example, for a 12 lead ECG waveform, a number of channels is 12, and atrous convolution is performed on each of the 12 input channels. Further, while the present example shows a single atrous convolutional layer 210, more than one atrous convolutional layer may be applied. For example, a number of atrous convolutional layers may be 2, 3, 4, or 5.

Next, downstream of the atrous convolutional layer 210, the neural network model 200 comprises an inverted residual and max pooling module 220. The inverted residual and max pooling module 220 is further described at FIG. 3A. Turning to FIG. 3A, the inverted residual and max pooling module 220 comprises of a plurality of inverted residual layers arranged in series. In this example model, three inverted residual layers 330, 350, and 370 are included. Additional inverted residual layers may be used, for example, to increase a number of channels. As a non-limiting example, a neural network model with greater number of inverted residual layers may be implemented when a number of input ECG channels is less than 12.

Further, max pooling operation may be performed on an output of one inverted residual layer before being input into a subsequent inverted residual layer until a last inverted residual layer is reached. Accordingly, the inverted residual and max pooling module 220 includes a max pooling layers 340 and 360.

Accordingly, the inverted residual layer 330 may include X number of bottleneck modules (each bottleneck module having an inverted residual structure with bottleneck layers flanking one or more intermediate expansion layers) with the operation of the inverted residual layer 330 repeated n times. Further, the inverted residual layer 350 may include Y number of bottleneck modules with the operation of the inverted residual layer 350 repeated m times, and the inverted residual layer 370 may include Z number of bottleneck modules.

Each inverted residual layer may include a number of bottleneck modules, each bottleneck module having an inverted residual structure wherein an input layer and an output layer are bottleneck layers with one or more intermediate expansion layers. A single bottleneck module 380 having an inverted residual structure is shown at FIG. 3B. The bottleneck module 380 includes an input bottleneck layer 382, an intermediate expansion layer 384, and an output bottleneck layer 386. A one dimensional depth-wise convolution is applied on the input layer; wherein lightweight filtering is performed by applying a single convolutional filter per input channel. Next, a 1×1 convolution across all channels, called a pointwise convolution, is applied at the expansion layer, through which ECG features across all the channels is integrated.

As shown an output number of channels (v) is greater than an input number of channels (u). Thus, through each bottleneck module, the number of input channels are gradually increased to allow for integration of information across ECG leads. For example, from an initial 12 input channels (one channel corresponding to each ECG lead), the input channels may be increased to 32, 16, 24, 40, 80, 112, 192, then 320 input channels before being input into the last 1D-convolutional layer (230 at FIG. 2). In this way, spatial and temporal higher resolution of the ECG features are preserved at each bottleneck module, and hence at each inverted residual layer. As a result, the higher resolution of the ECG features output from the initial atrous layer is preserved and rich representation of ECG features is obtained through the serial inverted residual layers.

Depending on a desired number of output channels, desired computational speed, and available computational capacity, X, Y, Z, m, and n may be adjusted. One embodiment of the inverted residue and max pooling module 220 is shown in FIG. 4A. In particular, FIG. 4A shows an inverted residual and max pooling module 420 comprising a first inverted residual layer 430 including two bottleneck modules, a second inverted residual layer including three bottle neck modules, and a third inverted residual layer including four bottleneck modules. Further, the operation of the inverted residual layer 430 and the inverted residual layer 450 are repeated two and three times respectively.

Another embodiment of the inverted residue and max pooling module 220 is shown in FIG. 4B. Specifically, FIG. 4B shows each of the first inverted residual layer 430, the second inverted residual layer 450 and the third inverted residual layer 470 including one bottleneck module. Further, similar to FIG. 4A, the operation of the inverted residual layer 430 and the inverted residual layer 450 are repeated two and three times respectively.

In one example, a computational model may store both neural network models, including a larger neural network model (e.g., with the architecture of neural network model 200) having an inverted residual and max pooling module at FIG. 4A and a smaller neural network model (also, having the architecture of neural network model 200) having an inverted residual and max pooling module at FIG. 4B. During a first condition when ECG signal from a fewer number of ECG leads (e.g., less than a threshold number of leads) are available, the processor may choose to utilize the first neural network model, and during a second condition, when ECG signal is available from a threshold number or greater than threshold number of leads, the processor may choose the second neural network model. In this way, risk prediction may be optimized using a computational model that includes more than one neural network model and choosing a model based on a quantity of ECG data available.

In another example, a neural network model that is chosen for risk prediction may be based on availability of computational resources. For example, the smaller model including the module at FIG. 4B may be used when run locally on a computer without a GPU; while the larger model including the module at FIG. 4A may be used when more computational resources are available.

In this way, the convolutional neural network model with initial atrous convolutions and residual connections with bottleneck layers predicts perioperative risk based on multi-lead input ECG data with increased accuracy. The neural network architecture with initial atrous convolutions and subsequent multi-channel 1D convolutions, reduces network size to less than 1/10^{th} the size of a regular convolutional neural network and 10 times faster runtime. Thus, the neural network model provides improved computational efficiency in addition to improved accuracy.

Further, by using an interpretable model (e.g., LIME) on the trained neural network model, most relevant features highlighted include abnormalities with the QRS complex, changes in amplitude associated with ventricular hypertrophy, changes in width associated with conduction blocks (left bundle branch block and right bundle branch block), and changes in onset (premature ventricular contractions and premature atrial contractions). Example highlights by LIME are shown in FIGS. 11A, and 11B. The above-mentioned features were most frequently highlighted in ECG waveforms from the precordial leads (leads closest to the heart) but also present in other leads as well. These features correspond to clinical evaluation of an ECG by an expert clinician. This additionally supports the accuracy of the convolutional neural network model described herein.

Returning to FIG. 2, subsequent to the inverted residual and max pooling module 220, is a one dimensional regular convolutional layer 230. Output from the convolutional layer 230 is average pooled via an average pooling layer 240 before passing through the fully connected layer 250. The fully connected layer 250 is connected to a decision layer 260. The decision layer may be a binary classification layer or a multi-class classification layer or a regression layer which may output a binary classification, multi-class classification, or a regression output respectively. Depending on the training dataset, the output 270 of the decision layer may predict a primary outcome of mortality or a secondary outcome of an adverse cardiac event as discussed above.

In one example, as discussed above, additionally, LIME may be used to identify and visualize relevant features in the ECG used for model decision making (FIGS. 11A, and 11B).

Next, FIG. 5 shows a high-level flow chart illustrating an example method 500 for evaluating perioperative risk using a convolutional neural network model, such as the neural network model at FIG. 2. Method 500 may be implemented by a processor, such as processor 104 computational model stored in non-transitory memory, such as non-transitory memory 106 of the ECG processing system 100, an edge device connected to the image processing system, a cloud in communication with the ECG processing system, or any appropriate combination thereof.

At 502, the method 500 includes acquiring ECG waveform data from an ECG system, such as the ECG system 120 at FIG. 1. In one example, the ECG system may be a clinical grade ECG system and may acquire 12 lead ECG data, which may be used for perioperative risk evaluation. As discussed above, in some examples, ECG data from less than 12 leads may be used. The ECG data may be acquired within a threshold period (e.g., 30 days) prior to a surgical procedure. In some examples, multiple ECG data may be acquired at different time points within the threshold period before the surgical procedure, and the results of the risk predictions may be evaluated in a cumulative manner. The surgical procedure maybe a cardiovascular surgical procedure or a non-cardiovascular surgical procedure. In some examples, the risk predictions may be performed for non-surgical procedures, such as minor procedures, chemotherapy, radiation therapy, etc.

In other examples, fewer than 12 lead ECG data may be used for perioperative risk prediction, and is within the scope of the disclosure.

Next, at 504, the method 500 includes pre-processing the acquired ECG data. In one example, the ECG data may be pre-processed according to pre-processing performed during training of the neural network model. The pre-processing may include filtering and normalization, for example. In some examples, pre-processing the ECG waveform data includes selecting a time window. The time window may be in a range between 10 seconds and 2 seconds, for example.

In one embodiment, the ECG waveform data is a time series data. In another embodiment, the ECG waveform data may be converted to image data during preprocessing and used as input into the neural network model.

Next, at 506, the pre-processed ECG data is input into a trained neural network model, such as the neural network model of FIG. 2 or FIG. 12. In particular, the trained neural network model may be trained to predict risk of one or more post-operative outcomes, including mortality and/or major cardiovascular event. Additional details of training the neural network model for post-operative risk prediction is described below.

Next, at 508, the method 500 includes outputting post-operative risk metric as output from the neural network model and may further include displaying the post-operative risk metric. In one example, the perioperative risk metric may be an indication of post-operative mortality. For example, the indication may be a degree of risk (e.g., high, intermediate, low) of post-operative mortality or any binary or multi-level classification of risk of post-operative mortality. In some examples, the post-operative risk metric for mortality may be a regression output providing a probability of risk (e.g., risk of post-operative mortality or risk of post-operative cardiac adverse event) for a given input ECG waveform.

In another example, the perioperative risk metric may be an indication of post-operative adverse cardiac events. For example, the indication may be a degree of risk (e.g., high, intermediate, low) of post-operative adverse cardiac event or any binary or multi-level classification of risk of post-operative mortality. The adverse cardiac events may include any of the following but not limited to myocardial infarction (MI), arrhythmias, cardiac arrest, heart block, and pulmonary edema. In some examples, the post-operative risk metric for adverse cardiac events may be a regression output providing a probability of risk (e.g., risk of post-operative mortality or risk of post-operative cardiac adverse event) for a given input ECG waveform.

In some examples, in addition to indicating risk of post-operative mortality and/or risk of post-operative MACE, a potential cause of post-operative mortality and/or a potential event of MACE (e.g., MI, arrhythmias, cardiac arrest, heart block, or pulmonary edema) may be indicated.

In some examples, a post-operative period during which the evaluated risk of post-operative mortality and/or the evaluated risk of post-operative MACE is applicable is indicated. As a non-limiting example, an output of the neural network may indicate that there is an 85% probability of post-operative mortality for a subject within 15 days from the date of surgery. As another non-limiting example, an output of the neural network may indicate that there is an 65% probability of post-operative MACE for a subject within 15 days and 30 days from the date of surgery.

In one example, a method for selecting a patient for a surgery comprises determining one or more post-operative risk metrics via a convolutional neural network model receiving a pre-operative ECG waveform data as input, the ECG waveform data acquired via an ECG sensor system, and responsive to determining the one or more post-operative risk metric below a threshold risk, selecting the patient for the surgery. In one example, the method further comprises performing the surgery on the patient. In one example, the one or more post-operative risk metrics include a post-operative mortality risk metric indicating a risk for post-operative mortality within a threshold post-surgery time duration, and/or a post-operative MACE risk metric indicating a risk for post-operative MCE within the threshold post-surgery time duration. In one example, the surgery may be an elective surgery or a non-elective surgery. Further, in one example, the convolutional neural network model includes at least one atrous convolutional layer and one or more inverted residual layers. Further, a one-dimensional convolution is performed in the one or more inverted residual layers. In one example, the pre-operative ECG data may be ECG data acquired from a 12-lead ECG system. In one example, the pre-operative ECG waveform data is time series data. In another example, the pre-operative ECG waveform data is image data obtained based on the time series data.

Referring now to FIG. 6, it shows an example method 600 for training a neural network model, such as neural network model 200 at FIG. 2. Method 600 may be implemented based on executable instructions stored in non-transitory memory, such as non-transitory memory 106 of the ECG processing system 100, an edge device connected to the image processing system, a cloud in communication with the ECG processing system, or any appropriate combination thereof.

At 602, the method 600 includes acquiring a training data set comprising a plurality of ECG waveforms. For example, the plurality of ECG forms may be obtained from a plurality of patients who had ECG obtained within a threshold period before a surgical procedure or a non-surgical procedure or non-surgical treatment. In one example the threshold period is 30 days before the procedure or treatment. Accordingly, the training dataset includes a plurality of ECGs obtained within 30 days before the procedure or treatment. In other examples, the threshold period may be greater than 30 days or less than 30 days. For example, the threshold period may be in a range from 1 day before the surgery to 1 year before the surgery. In some examples, the threshold period may be based on the type of procedure or treatment performed.

Further, the training dataset may be labelled with an observed post-operative outcome (e.g., mortality or MACE) during a post-operative period of the plurality of patients. In one example, the post-operative period may be a duration of hospitalization (prior to discharge from the hospital). Further, the observed post-operative outcome is based on a patient health record. In another example the post-operative period may be 30 days (including duration of hospitalization and duration after discharge). In some examples, the post-operative period may be in a range from 30 days to 90 days. In some examples, the post-operative period may be greater than 90 days.

In one embodiment, a first training data set labelled with primary outcome of mortality is used for the training the neural network model to predict risk of post-operative mortality, and a second training dataset labelled with secondary outcome of adverse cardiac events (which may be any of MI, arrhythmias, heart block, CIF and pulmonary edema, but not limited to the above) is used for training the neural network model independently to predict risk of post-operative adverse cardiac events. In some examples, two neural network models (having the same architecture as discussed at FIG. 2) may be trained and implemented separately to predict post-operative mortality and post-operative adverse cardiac events respectively.

An example dataset for training, testing, and validating a neural network model, such as neural network model 220 at FIG. 2 or FIG. 12, for post-operative risk prediction, may include ECG data acquired from patients who had a pre-operative ECG within 30 days prior to a surgical procedure. Example baseline characteristics and outcomes of the study participants (derivation cohort) are shown at Table 1 below.

At 604, the method 600 includes pre-processing the training data set. The pre-processing may include one or more of normalization by mean and standard deviation, and filtering (e.g., to remove very low frequency noise).

Next, at 606, the method 600 includes training the neural network model with the training data set. During training, the neural network model may be initialized with random weights and trained with a loss function of binary cross entropy. Stopping may be performed based on validation dataset's area under the receiver operating curve. Next, at 608, the method 600 includes validating using a validation dataset, and updating neural network hyperparameters. Last, at 610, the method includes testing the trained and validated model using a test data set.

In some examples, an interpretable model (e.g., based on LIME technique) may be implemented during the training for optimizing the neural network model parameters. For example, based on ECG features highlighted via the interpretable model, and based on comparison of the prediction during the training and the highlighted ECG features, model parameters (e.g., atrous convolution step size, dilation size, etc.) may be updated to improve model performance, in particular model accuracy in predicting perioperative risk.

Further, in one embodiment, a number of ECG leads for ECG acquisition may be determined based on features identified via the interpretable model. For example, if the one or more important features contributing to a perioperative risk prediction for post-operative mortality is highlighted in a set of leads, a number of ECG leads may be reduced to include the set of leads and the ECG data may be acquired from the reduced number of leads for perioperative risk prediction for mortality. In this way, the features identified via the interpretable model may be used to reduce the number of ECG leads used for ECG acquisition. Thus, with fewer than 12 leads, more accurate perioperative risk prediction may be achieved. As fewer leads are used, in some examples, this enables remote ECG acquisition for post-operative risk evaluation.

As a non-limiting example, as shown in FIGS. 11A and 11B, LIME highlights the QRS complexes as the most relevant features used for model decision making, and frequently highlights precordial premature contractions and intraventricular block on the precordial leads. Since ECG data from the pericordial leads contain the most relevant features, in one example, a number of leads may be reduced to include only the pericordial leads.

Referring to FIG. 11A, it shows graphs of 12 lead ECGs 1110, 1120, and 1130 including indications to most important ECG features contributing to the perioperative risk prediction for mortality. FIG. 11B shows 12 lead ECGs 1170, 1180, and 1190 including indications to most important ECG features contributing to the perioperative risk prediction for major cardiovascular events. As seen in FIGS. 11A and 11B, one or more ECG features that are relevant to the post-operative risk prediction are indicated. In the present examples, the one or more ECG features are indicated by oval indications. It will be appreciated that that other types of indications such as arrows pointing to the one or more ECG features, colored highlights of the one or more ECG features, other geometric indications (e.g., square, rectangle, triangle, polygonal boxes, etc.) may be used to indicate the one or more ECG features that have been determined to be relevant to the prediction. In some examples, a degree of relevance may be indicated with different colors. For example, different shades of a color may be used to indicate different degrees of relevance of the one or more ECG features that have been determined to be relevant. In some examples, different colors may correspond to different degrees of relevance. In some examples, the one or more ECG features may be indicated as enlarged portions separately from the input waveforms. In some other examples, as a cursor is positioned on the highlighted/indicated portion, the highlighted/indicated portion may be enlarged. In still further examples, the leads that include the one or more important features may be extracted and displayed separately in addition to (e.g., at a different portion of the user interface than the input waveform) or alternatively to the input waveform. In yet another example, the user may select the type of indication for display on the GUI.

In the present examples, the important ECG features (ECG portions indicated by oval boundary) contributing to presence of mortality risk and/or major cardiovascular event risk are frequently indicated primarily in the pericordial leads v1, v2, v3, v4, v5, and v6.

In this way, an interpretable model output based on LIME for each perioperative risk prediction by the trained neural network model includes highlights for one or more important ECG features that contributed to the corresponding prediction outcome. In the above example, the highlights are boxes over the one the ECG waveforms where the one or more important ECG features were identifies. In some examples, the highlighting boxes may include shading. In some example, the ECG waveforms corresponding to the one or more important ECG features may be indicated in a different color. In some examples, the relevant portions on the ECG graphs may be enlarged and displayed separately from the input ECG waveform and/or the risk prediction outcome. In still further examples, one or more leads from which data the one or more important ECG features were identified may be automatically determined and indicated to the user along with one or more of the input ECG waveform, the predicted outcome, and highlights of the one or more one or more important ECG features on the input ECG waveform.

In some examples, a degree of relevance of one or more ECG features may be indicated.

While the above examples illustrate using LIME, other model interpretation/explanation techniques, such as SHapley Additive exPlanations (SHAP), may be used and are within the scope of the disclosure.

In one example, a method for estimating and/or evaluating a post-operative outcome comprises receiving ECG waveform data from a set of ECG electrodes, inputting the ECG waveform data into a trained neural network model, and processing the ECG waveform data via the trained neural network model to output the estimation and/or evaluation of the post-operative outcome, wherein the trained neural network model is trained with a training dataset comprising a set of ECG waveforms from a set of patient population acquired within a threshold period before a corresponding procedure for each patient and labelled with a corresponding post-procedure outcome for each patient. In one example, the neural network model is the neural network model at FIG. 2 that includes an inverted residual and max pooling layer at FIG. 4A, may be trained to predict mortality outcomes with the input of one 12-lead ECG obtained within 30 days prior of the procedure. As discussed above, the neural network model comprises at least one atrous convolutional layer and a series of inverted residual layers.

Referring to FIG. 12, it shows a schematic illustration of an architecture of another example neural network model 1200 for perioperative risk prediction. Using a single ECG time series data as input, the neural network model 1200 outputs a post-operative risk outcome. In one example, a first neural network model is trained to output a primary outcome of post-operative mortality risk, and a second neural network model is trained to output a secondary outcome of composite major cardiac events. The first and second neural network models may be trained independently on the primary and secondary outcomes.

As discussed above with respect to FIGS. 2-4B, the input ECG waveform is preprocessed before passing through an atrous convolutional layer 1210. The atrous 1D convolutional layer 1210 is similar to the atrous convolutional layer 210. The initial atrous convolutional layer 1210 downsamples the pre-processed input ECG waveforms prior to downstream layers. Output of the atrous convolutional layer is passed through serial layers 1220, 1230, 1250, and 1270. Each of the serial layers have at least one bottleneck module having an inverted residual structure where the input and output are bottleneck layers with an intermediate expansion layer. Bottleneck modules in each of the serial layers are similar to bottleneck module described with respect to FIG. 3B. In each set of expansion layers with bottleneck layers preceding and succeeding, the number of input channels gradually increased to allow for integration of information across ECG leads. From initial 12 input channels representing each ECG lead, the input channels for each layer increased to 32, 16, 24, 40, 80, 112, 192, then 320 input channels before average pooling (1282) prior to the final fully connected layer (1284). For computational efficiency, each convolutional layer only relied on 1-dimensional convolutions while inflation of output channels allows for integration of information across leads. In this way, the neural network model for ECG interpretation and integration with clinical risk factors is utilized to predict the co-primary outcomes of post-operative mortality or composite major adverse cardiac events.

Additionally, in some examples, LIME may be used on 12-lead ECGs to identify and visualize relevant features in the ECG used for model decision making (FIGS. 11A and 11B). In the illustrated examples at 11A and 11B, LIME highlighted the QRS complexes as the most relevant features used for model decision making, and frequently highlighted precordial premature contractions and intraventricular block on the precordial leads. The most relevant features include abnormalities with the QRS complex including changes in amplitude associated with ventricular hypertrophy, changes in width associated with conduction blocks (left bundle branch block and right bundle branch block), and changes in onset (premature ventricular contractions and premature atrial contractions). This was most frequently highlighted in precordial leads (leads closest to the heart) but also present in other leads as well. In one example, LIME may be used with 1000 samples per study to identify relevant features in the ECG waveform by iteratively randomly perturbing 0.5% of the waveform and identifying which changes most impacted model performance.

The neural network model 1200 shows demonstrated improvement in accuracy in predicting mortality and cardiovascular complications, as evidenced by experimental data shown below. Thus, the neural network model 1200 is a highly efficient deep learning architecture with fewer parameters and requiring less computational power to train, up to 100× smaller than other previously published architectures, suggesting the particular efficaciousness of its specific architecture. The improvement in speed-up and computational efficiency allows for the model to be run solely on a standard CPU in 0.078 seconds at inference time.

A particular strength of the neural network model is the relative few number of parameters and high computational efficiency compared to previously published neural network architectures for interpreting ECG without a loss in predictive performance, therefore being more computational efficient than previously published models. Table 9 below shows reduced computational complexity of the neural network model compared to other deep learning models. The neural network models discussed herein can be deployed without GPUs, which are less common in clinical settings, and still take less than 0.003 seconds to run inference on one ECG.

The methods and systems described herein present an important step towards streamlining perioperative risk evaluation. Accordingly, technical advantages of utilizing the neural network models described herein include increased accuracy in evaluating perioperative risk. Further, the perioperative risk evaluation may be performed in an expedited manner using a single ECG dataset for a patient. Furthermore, in some examples, the ECG dataset may include 12 lead data. In some other examples, data from fewer than 12 leads may be utilized.

In one representation, provided herein is a method for performing risk assessment, the method comprising receiving an electrocardiogram (ECG) waveform data acquired via an ECG sensor system; inputting the ECG waveform data in to a trained neural network model, the trained neural network model comprising an initial convolutional layer, the initial convolutional layer applying an atrous convolutional operation to the ECG waveform data; and determining a degree of risk of a post-operative cardiac health condition according to an output of the trained neural network model; wherein the trained neural network model is trained using a training dataset comprising a plurality of pre-operative ECG waveforms linked with corresponding post-operative cardiovascular outcomes; and wherein the plurality of pre-operative ECG waveforms are acquired within a pre-operative period before respective medical procedures. In a first example of the method, the ECG waveform data includes ECG waveforms acquired from a number of ECG leads of the ECG sensor system; and wherein the number of ECG leads is any of 2, 3, 4, 5, 6, 7, 8, 9, 10, 11, or 12 leads. In a second example of the method, which optionally includes the first example, the post-operative cardiac health condition is any of a post-operative mortality, a cardiac arrest, a myocardial infarction, a heart block, or a pulmonary edema. In a third example of the method, which optionally includes one or more of the first and the second examples, the trained neural network comprises a series of inverted residual layers following the initial atrous convolutional layer; and wherein the series of inverted residual layers are configured to increase a number of input channels gradually from one inverted residual layer to a subsequent inverted residual layer. In a fourth example of the method, which optionally includes one or more of the first through third examples, each of the inverted residual layers comprises one or more bottleneck modules, each of the one or more inverted residual layers having at least one bottleneck module with an inverted residual structure comprising an expansion layer in between a preceding bottleneck layer and a succeeding bottleneck layer. In a fifth example of the method, which optionally includes one or more of the first through fourth examples, determining the risk of the post-operative cardiac health condition via the trained neural network model includes processing the input ECG data to output one or more ECG features, wherein the one or more ECG features include one or more ECG waveform changes corresponding to one or more of ventricular hypertrophy, conduction blocks, premature ventricular contractions and premature atrial contractions. In a sixth example of the method, which optionally includes one or more of the first through fifth examples, the one or more ECG waveform changes include changes in QRS complex including one or more of amplitude changes, width changes, and onset changes. In a seventh example of the method, which optionally includes one or more of the first through sixth examples, the method further comprises indicating the risk of the post-operative cardiac health condition and the ECG features via a user interface. In an eighth example of the method, which optionally includes one or more of the first through seventh examples, the trained neural network has an area under the curve (AUC) of greater than 0.75. In a ninth example of the method, which optionally includes one or more of the first through eighth examples, the medical procedure is selected from the group consisting of a cardiac surgery, a non-cardiac surgery, an endoscopic procedure, a catheterization lab procedure, a non-surgical procedure, a radiation therapy, an immunotherapy, a chemotherapy, and any combination thereof.

In another representation, provided herein is a method for selecting a patient for a surgical procedure, the method comprising: receiving ECG waveform data acquired via a ECG sensor system attached to the patient; inputting the ECG waveform data into a trained neural network model; obtaining one or more post-operative risk metrics for one or more adverse post-operative outcomes based on the trained neural network model; and selecting the patient for the surgery based on the one or more post-operative risk metric; wherein the one or more adverse post-operative outcomes comprises one or more of a post-operative mortality, a cardiac arrest, a myocardial infarction, a heart block, and a pulmonary edema; and wherein the trained neural network model includes at least one atrous convolutional layer performing an atrous convolutional operation on the ECG waveform data. In one example of the method, the method further comprises performing the surgical procedure on the patient. In a second example of the method which optionally includes the first example, the trained neural network model is trained using a training dataset comprising a plurality of pre-operative ECG waveforms linked with corresponding post-operative adverse outcomes; and wherein the plurality of pre-operative ECG waveforms are acquired within a pre-operative period before respective surgical procedures. In a third example of the method, which optionally includes one or more of the first and the second examples, the trained neural network comprises a series of inverted residual layers following the initial atrous convolutional layer; and wherein the series of inverted residual layers are configured to increase a number of input channels gradually from one inverted residual layer to a subsequent inverted residual layer. In a fourth example of the method, which optionally includes one or more of the first through third examples, each of the inverted residual layers comprises one or more bottleneck modules, each of the one or more inverted residual layers having at least one bottleneck module with an inverted residual structure comprising an expansion layer in between a preceding bottleneck layer and a succeeding bottleneck layer. In a fifth example of the method, which optionally includes one or more of the first through fourth examples, the trained neural network has an area under the curve (AUC) of greater than 0.75.

In one implementation, a computer-implemented method for performing risk assessment before an expected procedure or treatment for a patient comprises receiving, at a processor of a computer, an electrocardiogram (ECG) waveform data acquired from an ECG sensor system; and processing, via the processor, the ECG waveform data using a trained neural network model to generate a one or more of a first indication of a risk of mortality after the expected procedure or treatment, and a second indication of a risk of MACE after the expected procedure or treatment; and displaying, via a display coupled to the processor, the first output; wherein the trained neural network model is trained using a first training dataset comprising a plurality of ECG waveforms, each of the plurality of ECG waveforms acquired before a respective procedure within a first time period and labelled with a corresponding post-procedure mortality status within a second time period after the respective procedure; and wherein the trained neural network model is further trained using a second training dataset comprising a second plurality of ECG waveforms, each of the second plurality of ECG waveforms acquired before a respective procedure within the first time period and labelled with a corresponding post-procedure outcome related to the one or more major adverse cardiac events within the second time period after the respective procedure. In a first example of the method, the trained neural network includes at least one atrous convolutional layer performing an atrous convolutional operation on the ECG waveform data. In a second example, which optionally includes the first examples, the one or more post-procedure major adverse cardiac events include one or more of a cardiac arrest, a myocardial infarction, a heart block, and a pulmonary edema. In a third example of the method, which optionally includes one or more of the first and second examples, the method further comprises applying a model interpretable function to the first output and the second output from the trained neural network model and determining one or more relevant ECG features relevant to the first and the second outputs based on the model interpretable function; and outputting, via the display, the one or more relevant ECG features via a user interface. In a fourth example of the method, which optionally includes one or more of the first through third examples, the model interpretable function is configured according to a specified Local Interpretable Model-Agnostic Explanations (LIME) model for ECG waveform data. In a fifth example of the method, which optionally includes one or more of the first through fourth examples, the one or more relevant ECG features include one or more ECG waveform abnormalities indicated in ECG waveform data from the ECG sensor system. In a sixth example of the method, which optionally includes one or more of the first through seventh examples, the one or more relevant ECG features include one or more abnormalities in one or more QRS complexes indicated in one or more leads, the one or more abnormalities including changes in amplitude of the QRS complexes, changes in width of the QRS complexes, and changes in onset of the QRS complexes. In a seventh example of the method, which optionally includes one or more of the first through sixth examples, the one or more leads are precordial leads. In an eighth example of the method, which optionally includes one or more of the first through seventh examples, the expected procedure or treatment is a non-surgical procedure. In a ninth example of the method, which optionally includes one or more of the first through eighth examples, the expected procedure is a surgical procedure. In a tenth example of the method, which optionally includes one or more of the first through ninth examples, the surgical procedure is selected from the group consisting of procedures listed in table 2 below.

In one embodiment, a system for performing pre-operative risk assessment comprises at least one memory storing a trained neural network model and executable instructions; at least one processor communicably coupled to the at least one memory and when executing the instructions cause the processor to: receive electrocardiogram (ECG) waveform data of a patient, the ECG waveform data acquired via an ECG sensor system and acquired within a threshold period before a desired procedure; input the ECG waveform data in to the trained neural network model; obtain as output from the trained neural network model, one or more post-operative risk metrics; and display, via a display portion of a user interface coupled to the at least one processor, the one or more post-operative risk metrics. In one example of the system, the trained neural network model is trained using a set of ECG waveform data, the set of ECG waveform data acquired from a patient population and within the threshold period before corresponding medical procedures and labelled with corresponding post-operative outcomes for mortality within a post-operative period. In a second example of the system, which optionally includes the first example, the trained neural network model is trained using a second set of ECG waveform data, the second set of ECG waveform data acquired from a patient population within the threshold period before corresponding medical procedures and labelled with corresponding post-operative outcomes for one or more non-fatal major adverse cardiac events within a post-operative period. In a third example of the system, which optionally includes one or more of the first and the second examples, the one or more post-operative risk metric includes one or more of a post-operative mortality risk score and a post-operative MACE risk score; and wherein the one or more post-operative major adverse cardiac events includes one or more of a cardiac arrest, a myocardial infarction, a heart block, and a pulmonary edema. In a fourth example of the system, which optionally includes one or more of the first through third examples, the trained neural network comprises at least one atrous convolutional layer. In a fifth example of the system, which optionally includes one or more of the first through fourth examples, the desired procedure is selected from the group consisting of a cardiac surgery, a non-cardiac surgery, an endoscopic procedure, a catheterization lab procedure, a non-surgical procedure, a radiation therapy, an immunotherapy, a chemotherapy, and any combination thereof. In a sixth example of the system, which optionally includes one or more of the first through fifth examples, the at least one atrous convolutional layer is an initial layer of the trained neural network model and receives the normalized ECG waveform data as input. In a seventh example of the system, which optionally includes one or more of the first through sixth examples, the trained neural network model comprises a plurality of inverted residual layers downstream of the at least one atrous convolutional layer, each of the plurality of the inverted residual layers comprising one or more bottleneck modules, each of the one or more bottleneck modules configured to output a second number of channels greater than a first number of channels input into each of the one or more bottleneck modules. In an eighth example of the system, which optionally includes one or more of the first through seventh examples, the trained neural network model further comprises a non-atrous convolutional layer receiving input from a last inverted residual layer, and a fully connected layer receiving an average pooled input from the non-atrous convolutional layer; and wherein the non-atrous convolutional layer applies a one dimensional non-atrous convolutional operation on its input. In a ninth example of the system, which optionally includes one or more of the first through eighth examples, the at least one memory stores further instructions that when executed cause the processor to: apply a model interpretable function to the ECG waveform data one or more post-operative risk metrics output by the trained neural network model; determine one or more relevant ECG features contributing to the one or more post-operative risk metrics based on the model interpretable function; and indicate the one or more relevant ECG features via the user interface. In a tenth example of the system, which optionally includes one or more of the first through ninth examples, the model interpretable function is configured according to a specified Local Interpretable Model-Agnostic Explanations (LIME) model for the ECG waveform data. In a eleventh example of the system, which optionally includes one or more of the first through tenth examples, the at least one memory stores further instructions that when executed cause the processor to: determine whether the patient is recommended for the intended procedure based on the one or more post-operative risk metrics; and output the recommendation via the user interface. In a twelfth example of the system, which optionally includes one or more of the first through eleventh examples, the ECG waveform data is a time-series data acquired via the ECG sensor system.

In another embodiment, a method for performing pre-operative risk assessment for a patient comprises receiving, at a processor, an electrocardiogram (ECG) waveform data acquired from an ECG sensor system; and processing, via the processor, the ECG waveform data using a trained neural network model to generate a first output including a risk of post-operative mortality; and displaying, via a display coupled to the processor, the first output; wherein the trained neural network model is trained using a first training dataset comprising a plurality of ECG waveforms, each of the plurality of ECG waveforms acquired before a respective procedure within a pre-operative period and labelled with a corresponding post-operative mortality status within a threshold duration after the respective procedure. In one example of the method, the method further comprises processing, via the processor, the ECG waveform data using the trained neural network model to generate a second output including a risk of one or more post-operative non-fatal major adverse cardiac events; and wherein the trained neural network model is further trained using a second training dataset comprising a second plurality of ECG waveforms, each of the second plurality of ECG waveforms acquired before a respective procedure within the pre-operative period and labelled with a corresponding post-operative outcome related to the one or more major adverse cardiac events within the threshold duration after the respective procedure. In a second example of the method, which optionally includes the first example, the trained neural network includes at least one atrous convolutional layer performing an atrous convolutional operation on the ECG waveform data. In a third example, which optionally includes one or more of the first and the second examples, the one or more post-operative major adverse cardiac events include one or more of a cardiac arrest, a myocardial infarction, a heart block, and a pulmonary edema. In a fourth example of the method, which optionally includes one or more of the first through third examples, the method further comprises applying a model interpretable function to the first output and the second output from the trained neural network model and determining one or more relevant ECG features relevant to the first and the second outputs based on the model interpretable function; and outputting, via the display, the one or more relevant ECG features via a user interface. In a fifth example of the method, which optionally includes one or more of the first through fourth examples, the model interpretable function is configured according to a specified Local Interpretable Model-Agnostic Explanations (LIME) model for ECG waveform data. In a sixth example of the method, which optionally includes one or more of the first through fifth examples, the one or more relevant ECG features include one or more ECG waveform abnormalities indicated in ECG waveform data from the ECG sensor system. In a seventh example of the method, which optionally includes one or more of the first through sixth examples, the one or more relevant ECG features include one or more abnormalities in one or more QRS complexes indicated in one or more leads, the one or more abnormalities including changes in amplitude of the QRS complexes, changes in width of the QRS complexes, and changes in onset of the QRS complexes. In an eighth example of the method, which optionally includes one or more of the first through seventh examples, the one or more leads are precordial leads.

In another embodiment, a method for performing pre-operative risk assessment for a patient comprises receiving, at a processor, an electrocardiogram (ECG) waveform data acquired from an ECG sensor system, the ECG waveform data acquired within a pre-operative period prior to a medical procedure or treatment; and processing, via the processor, the ECG waveform data using a first trained neural network model to generate a first output including a risk of post-operative mortality; processing, via the processor, the ECG waveform data using a second trained neural network model to generate a second output including a risk of one or more non-fatal major adverse cardiac events; and displaying, via a display coupled to the processor, the first output and the second output; wherein the first trained neural network model is trained using a first training dataset comprising a plurality of ECG waveforms, each of the plurality of ECG waveforms acquired within the pre-operative period before a respective procedure or treatment, and labelled with a corresponding post-operative mortality outcome; and wherein the second trained neural network model is trained with a second training dataset comprising a plurality of ECG waveforms acquired within the pre-operative period before a respective procedure or treatment, and labelled with a corresponding post-operative major adverse cardiac event outcome. In a first example of the method, the method further comprises determining, via the processor, one or more first relevant ECG features of the ECG waveform data based on a model interpretability function, the one or more first relevant ECG features contributing to the first output; and determining, via the processor, one or more second relevant ECG features of the ECG waveform data based on the model interpretability function, the one or more second relevant ECG features contributing to the second output. In a second example of the method, which optionally includes the first example, each of the first and the second neural network models comprise an atrous convolutional layer receiving the ECG waveform data as input. In a third example of the method, which optionally includes one or more of the first and the second examples, the one or more post-operative major adverse cardiac events include one or more of a cardiac arrest, a myocardial infarction, a heart block, and a pulmonary edema. In a fourth example of the method, which optionally includes one or more of the first through third examples, the ECG waveform data is a time series data. In a fourth example of the system, which optionally includes one or more of the first through third examples, the medical procedure or treatment is selected from the group consisting of a cardiac surgery, a non-cardiac surgery, an endoscopic procedure, a catheterization lab procedure, a non-surgical procedure, a radiation therapy, an immunotherapy, a chemotherapy, and any combination thereof. In a fifth example of the method, which optionally includes one or more of the first through fourth examples, the first and the second neural network models each have an area under the curve (AUC) of 0.75 or greater than 0.75. In a sixth example of the method, which optionally includes one or more of the first through fifth examples, the method further comprises determining whether the patient is recommended to undergo the medical procedure or treatment based on one or more of the first and the second outputs, and displaying the recommendation on the display.

### Example: Experimental Data

The following set of experimental data is provided to better illustrate the claimed invention and is not intended to be interpreted as limiting the scope.

As discussed above, pre-operative risk assessments (also referred to herein as perioperative risk assessments) used in clinical practice are limited in their ability to identify risk for post-operative mortality and/or cardiac complications. The inventors herein have identified that electrocardiograms contain hidden risk markers that can help prognosticate post-operative mortality and/or non-fatal cardiac adverse events. In order to discriminate post-operative mortality or post-operative cardiac adverse events, the inventors have developed a neural network model trained to leverage waveform signals from pre-operative ECGs and output estimates of post-operative adverse outcomes. The example neural network model utilized for perioperative risk prediction in the results below is referred to as a deep learning model. The network architecture of the deep learning model is shown at FIG. 12. Using a single ECG time series data as input, the deep learning model outputs a post-operative risk outcome. In one example, a first deep learning model is trained to output a primary outcome of post-operative mortality risk, and a second deep learning model is trained to output a secondary outcome of composite major cardiac events. The first and second deep learning models are trained independently on the primary and secondary outcomes.

In the example discussed below, a derivation cohort of 45,969 pre-operative patients (age 59±19 years, 55% women) was used to develop the deep learning model to discriminate post-operative adverse outcomes from ECG waveform data acquired within 30 days before a surgical procedure. Model performance was assessed in a holdout internal test dataset and in two external hospital cohorts and compared with the Revised Cardiac Risk Index (RCRI) score. The experimental data is described in detail below.

The results (discussed further below) show that the deep learning model discriminates mortality with an AUC of 0.83 (95% CI 0.79-0.87) surpassing the discrimination of the RCRI score with an AUC of 0.67 (CI 0.61-0.72) in the held out test cohort. Patients determined to be high risk by the deep learning model's risk prediction had an unadjusted odds ratio (OR) of 8.83 (5.57-13.20) for post-operative mortality as compared to an unadjusted OR of 2.08 (CI 0.77-3.50) for post-operative mortality for RCRI>2. The deep learning model performed similarly for patients undergoing cardiac surgery with an AUC of 0.85 (CI 0.77-0.92), non-cardiac surgery with an AUC of 0.83 (0.79-0.88), and catherization/endoscopy suite procedures with an AUC of 0.76 (0.72-0.81). The algorithm similarly discriminated risk for mortality in two separate external validation cohorts from independent healthcare systems with AUCs of 0.79 (0.75-0.83) and 0.75 (0.74-0.76) respectively.

The results demonstrate how the deep learning algorithms discussed herein based on a neural network architecture, such as the neural network architectures at FIGS. 2, 3A, 3B, 4A, 4B, and 12, applied to pre-operative ECGs, can improve discrimination of post-operative adverse outcomes in a quick and efficient manner. The experimental data shown below demonstrate a comprehensive study of the example deep learning model shown at FIG. 12 and trained on perioperative ECGs. The performance of the deep learning model was evaluated on cohorts from three independent healthcare systems.

### Derivation and Internal Validation Cohort

The derivation cohort was derived from all patients undergoing inpatient procedures at Cedars-Sinai Medical Center (CSMC) between Jan. 1, 2015 and Dec. 31, 2019. 153,465 patients were identified aged 18 years or older who underwent 261,328 procedures in the operating room, catheterization laboratory, and endoscopy suite during the study period for model training and performed independent analyses for patients undergoing major surgeries and catheterization/endoscopy suite procedures. Of this source cohort, 45,969 patients were identified who had a complete ECG waveform image available for at least one 12-lead ECG performed within 30 days prior to the procedure date. These patients contributed 112,794 ECGs preceding 59,975 procedures to the primary analyses (FIG. 9). FIG. 8 shows an example timeline. A pre-operative period (between t0 and t2) when the pre-operative ECG waveforms were obtained is shown. Further, a post-operative period (t2-t3) during which post-operative outcomes of mortality and MACE are noted is also shown.

From CSMC, patients were randomly split 8:1:1 into a subset of 36,839 patients (contributing 90,633 ECGs) for training, 4,549 patients (contributing 11,217 ECGs) for internal validation, and the remaining 4,581 patients (contributing 10,944 ECGs) for final algorithm test analyses. All ECG waveform data were acquired through the clinical enterprise data warehouse at a sampling rate of 500 Hz and extracted as 10 second, 12×5000 matrices of amplitude values. ECGs with missing leads were excluded from analyses. Associated relevant clinical data for each patient were also obtained from the electronic health record.

### Clinical and Outcome Assessments

Patient demographic, clinical, and outcomes data were assessed from the electronic health record at the time of each procedure. From these data, the pre-operative clinical characteristics needed for calculating the revised cardiac risk index (RCRI) were identified, including: coronary artery disease, congestive heart failure, stroke or transient ischemia attack, pre-operative insulin use, creatinine greater than 2 mg/dL, and elevated risk surgery as defined by American College of Cardiology and American Heart Association guidelines. For the main analysis outcome was death during the hospitalization or during readmissions within 30 days.

As a secondary analysis, the deep learning model's performance in evaluating a composite outcome of MACE that included non-fatal major adverse cardiovascular events, defined by presence of post-operative myocardial infarction, cardiac arrest, heart block, pulmonary edema, as well as mortality was determined. Procedural complications were identified using relevant post-operative diagnoses that were present for the first time after procedure date or at discharge. Outcomes were adjudicated up to 30 days after date of procedure, with multiple procedures having independent outcomes windows based on procedure date. If the same outcome fell within the 30-day window for multiple procedures, the outcome was attributed to each of the procedures. Diagnoses were encoded by International Classification of Diseases (ICD)-9 or ICD-10 codes, and MACE was identified. A random subset of 100 patients with MACE were manually evaluated by chart review. MACE adjudication was correct in 88 (88%) patients. 81 (81%) of clinician evaluated RCRI scores were the same as the EHR diagnosis code based RCRI score. The mean RCRI score by manual review was 1.46, compared to 1.34 by automated evaluation, a mean difference of 0.13 between diagnoses present in clinical notes but not present in ICD9 codes.

### Electrocardiogram Assessments

A deep learning model was trained and validated to identify risk for death or post-operative cardiovascular events based on waveform signals from a single pre-operative 12-lead ECG. A schematic illustration of the deep learning model is shown at FIG. 12. The input of the model was a 12-lead ECG obtained within 30 days prior to an operative procedure, and the output were the hospitalization level outcomes following that procedure. Patients with multiple procedures were treated independently during model training, with each ECG paired with the most proximal subsequent procedure. To maximize sample size, all ECGs in the window of interest were utilized in the training set as training examples. In the validation and test cohorts, the analysis was limited to a single ECG most proximal to the procedure of interest for an individual patient to mimic how the model would be applied in clinical practice. When used, clinical features were input into the last fully connected layer prior to model output. Models were trained using the PyTorch deep learning library.

### Deep Learning Model Development

The deep learning model was designed to analyze 12-lead ECG waveform data starting with atrous convolutions followed by subsequent multi-channel 1D convolutions. As demonstrated further below, the deep learning model discussed herein, such the neural network models at FIGS. 2, 3A, 3B, 4A, 4B, and 12, improves model runtime and reduces model complexity while providing improved efficiency and accuracy in risk prediction compared to previously described architectures (See Tables 8 and 9 below). After initial atrous layers, the deep learning model incorporates convolutional layers with an inverted residual structure where the input and output are bottleneck layers with an intermediate expansion layer. In each set of expansion layers with bottleneck layers preceding and succeeding, the number of input channels gradually increased to allow for integration of information across ECG leads. The deep learning model had input only of the 12-lead ECG waveform data, and comparison models included clinical data including age and sex in the penultimate fully connected layers.

The deep learning model was initialized with random weights and trained with a loss function of binary cross entropy for 100 epochs using an ADAM optimizer with an initial learning rate between 5e-3 and 1e-4. Early stopping was performed based on validation dataset's area under the receiver operating curve. The atrous convolution's dilation and step size was grid-searched by hyperparameter tuning for optimal AUC with all other hyperparameters held constant (FIGS. 7A and 7B). Local Interpretable Model-agnostic Explanations was used to identify and visualize relevant features in the ECG used for model decision making.

Statistical Analyses within the Internal Test Cohort:

### Evaluation of Model Performance and Comparison to an Established Risk Calculator

Following model development in the training and validation sets, the ability of the deep learning model to discriminate the primary outcome of post-operative mortality in the held-out test set was assessed using the area under the curve (AUC) of the receiver operating characteristic curve (ROC). To understand the models performance in key patient populations, independent analyses were performed limited to patients undergoing cardiac surgery, non-cardiac surgery, interventional endoscopy suite or catheterization laboratory procedures, and patients with known cardiovascular disease or undergoing intermediate or high-risk surgeries. The predictions of the deep learning model were compared in the held-out test dataset with an established risk calculator (RCRI score), conventional ECG measures and interpretations, and alternate algorithms based on ECG measurement data.

The performance of the deep learning model was compared with that of an RCRI score >2, a commonly used threshold to identify high perioperative risk associated with a MACE rate greater than 8% in meta-analyses. A risk threshold of top 15% vs. bottom 85% of training set as high vs. low risk deep learning model prediction was identified to be calibrated similarly to an RCRI score threshold of 2. Odds ratios, sensitivity, and specificity for post-operative mortality and MACE were estimated at this threshold. Secondary analyses with MACE as the outcome were also performed. The continuous and categorical net reclassification index (NRI) for mortality and MACE rate associated with the addition of deep learning model to an RCRI score >2 was calculated. 10,000 bootstrapped samples were used to obtain 95^{th} confidence intervals for each estimate. To discern the potentially most informative features of the ECG waveform, in the context of comparing performance to that of the RCRI, 0.5% of the waveform for 1000 samples per study were iteratively randomly perturbed to identify which changes most impacted model performance. Implementation timings were evaluated using Python's timeit module.

### Comparison to Conventional and Alternate ECG Measures

Deep learning model performance was compared with that of a non-deep learning model (eXtreme Gradient Boost (XGB)) which considers the clinical risk factors used in the RCRI score (history of ischemic heart disease, congestive heart failure, cerebrovascular disease, insulin use, preoperative creatinine greater than 2 mg/dL, or elevated risk procedure). The deep learning model performance was also compared with that of models trained on the human interpretable features including: (i) clinical variables from the RCRI score; (ii) age and all ECG measurements obtained from MUSE including heart rate, axis, and intervals, and physician ECG interpretations; and, (iii) the combination of age, ECG measurements, physician ECG interpretations, and clinical variables. Physician interpretations in the clinical over-read section of MUSE were coded as distinct categorical variables and considered as potential predictors in comparison models as shown in Table 1. In particular, the non-deep learning models were trained with ECG measurements obtained from MUSE including heart rate, axis, and intervals (column 1) and physician ECG interpretations from the clinical over-read section of MUSE as categorical variables (column 2).

TABLE 1
ECG Measurements and Interpretations
used in non-deep learning models.

| Muse Measurements | Common Physician Interpretations |
|---|---|
| AtrialRate | Normal sinus rhythm |
| PAxis | Prolonged QT interval |
| POffset | Left axis deviation |
| POnset | Inferior infarct |
| PRInterval | Right bundle branch block |
| PharmaPPinterval | 1st degree A-V block |
| PharmaRRinterval | Suggest repeat electrocardiogram |
| QOffset | Nonspecific T wave abnormality |
| QOnset | Left atrial enlargement |
| QRSCount | Premature ventricular complexes |
| QRSDuration | Otherwise normal ECG |
| QTCorrected | No significant change was found |
| QTInterval | Low voltage QRS |
| QTcFrederica | Premature atrial complexes |
| RAxis | T wave abnormality, consider lateral ischemia |
| TAxis | Left Ventricular Hypertrophy |
| TOffset | Borderline ECG |
| VentricularRate | Anterior infarct |
| HeightCM | Septal infarct |
| WeightKG | Left anterior fascicular block |
| PatientAge | Nonspecific ST and T wave abnormality |

### External Validation of the Deep Learning Model for Discrimination of Post-Operative Mortality

To assess the model's performance in other hospital settings, the deep learning model was applied without any additional further fine tuning or training to patients from two separate external healthcare systems. To maximize rigor of external validation, patient data was not transferred across institutions: rather, investigators from each external institution independently collected outcomes information and ran model inference on their datasets to report summary statistics. The Stanford Healthcare (SHC) cohort included 101,375 patients contributing 162,540 pre-operative ECGs from May 1, 2007 to Jun. 30, 2018. All clinical characteristics and outcomes data were provided through the Stanford Research Data Repository Observational Medical Outcomes Partnership (OMOP) common data model. ECG waveform data was provided through the TraceMaster (Philips Healthcare) data management system and preprocessed with a low pass filter to further correct for wandering baselines and normalization of waveform data. The Columbia University Medical Center (CUMC) cohort included 9,028 patients contributing 9,028 pre-operative ECGs from Jan. 1, 2020 to Mar. 31, 2020. At CUMC, clinical characteristics and outcomes data were obtained from the clinical enterprise data warehouse and ECG waveform data was obtained from Muse (GE Healthcare) data management system. For each of the two external cohorts, the AUC for post-operative in-hospital mortality from analyses of a single pre-operative ECG was calculated. Procedures were linked to the most proximal preceding ECG performed within 30 days prior and post-operative mortality was assessed as mortality within 7 days after the procedure or during the hospitalization. The most common procedures are summarized in Table 2 below.

TABLE 2
Most common procedures based on frequency of pre-procedural ECGs by institution

| CSMC | SHC | CUMC |
|---|---|---|
| CVIC-PCI | RESECTION OF APICAL LUNG | CORONARY ARTERY |
| | TUMOR (EG, PANCOAST | BYPASS GRAFT |
| | TUMOR), INCLUDING CHEST | (CORONARY ARTERY |
| | WALL RESECTION, RIB(S) | BYPASS GRAFT) |
| | RESECTION(S), | |
| | NEUROVASCULAR | |
| | DISSECTION, WHEN | |
| | PERFORMED; WITH CHEST | |
| | WALL RECONSTRUCTION | |
| CVIC-AORTIC VALVE | INCISION AND DRAINAGE, | INSERTION, |
| REPLACEMENT/ | OPEN, OF DEEP ABSCESS | VENTRICULAR ASSIST |
| TRANSFEMORAL | (SUBFASCIAL), POSTERIOR | DEVICE |
| | SPINE; CERVICAL, THORACIC, | |
| | OR CERVICOTHORACIC | |
| AORTIC VALVE | PERCUTANEOUS VERTEBRAL | REPLACEMENT, AORTIC |
| REPLACEMENT | AUGMENTATION, INCLUDING | VALVE |
| | CAVITY CREATION (FRACTURE | |
| | REDUCTION AND BONE BIOPSY | |
| | INCLUDED WHEN PERFORMED) | |
| | USING MECHANICAL DEVICE, 1 | |
| | VERTEBRAL BODY, | |
| | UNILATERAL OR BILATERAL | |
| | CANNULATION (E.G., | |
| | KYPHOPLASTY); THORACIC | |
| CORONARY ARTERY | DISCECTOMY OF SPINE | CHOLECYSTECTOMY, |
| BYPASS GRAFTING | | LAPAROSCOPIC |
| MITRAL VALVE REPAIR | TOTAL KNEE REPLACEMENT | CREATION, |
| MINIMALLY INVASIVE | | TRACHEOSTOMY |
| ROBOTIC | | |
| MITRAL VALVE | FUSION OR REFUSION OF 2-3 | REPAIR OR |
| REPLACEMENT | VERTEBRAE | REPLACEMENT, |
| | | MITRAL VALVE |
| CVIC-PACEMAKER | OTHER EXPLORATION AND | CREATION, |
| IMPLANT DUAL | DECOMPRESSION OF SPINAL | TRACHEOSTOMY, OPEN |
| | CANAL | AMPUTATION, BELOW |
| | | KNEE |
| HIP ARTHROPLASTY | TOTAL REPLACEMENT OF HIP | ARTHROPLASTY, HIP, |
| TOTAL | | TOTAL |
| CYSTOSCOPY | EXCISION OR DESTRUCTION OF | INSERTION, SHUNT, |
| | OTHER LESION OR TISSUE OF | VENTRICULOPERITONEAL |
| | HEART, ENDOVASCULAR | |
| | APPROACH | |
| GI- | PERCUTANEOUS VERTEBRAL | STENT DES - |
| ESOPHAGOGASTRO- | AUGMENTATION, INCLUDING | CORONARY |
| DUODENOSCOPY WITH | CAVITY CREATION (FRACTURE | |
| PERCUTANEOUS | REDUCTION AND BONE BIOPSY | |
| ENDOSCOPIC GASTRO- | INCLUDED WHEN PERFORMED) | |
| JEJUNOSTOMY TUBE | USING MECHANICAL DEVICE, 1 | |
| PLACEMENT | VERTEBRAL BODY, | |
| | UNILATERAL OR BILATERAL | |
| | CANNULATION (E.G., | |
| | KYPHOPLASTY); LUMBAR | |

### Results

### Derivation Cohort Characteristics

The derivation cohort of 45,969 patients underwent 59,975 inpatient procedures between years 2013 and 2019 (FIG. 9). The mean age at the time of pre-operative ECG was 65.1±15.9 years, 4500 were women, and 22% had pre-existing coronary artery disease as shown in Table 3 below. Within the derivation cohort, there were 1,065 (1.80%) subsequent deaths and 1,730 (2.90%) post-procedural MACE events during the hospitalization. Clinical characteristics and index hospitalization outcomes are also shown in Table 3 for the training, internal validation, and test subsets of the derivation cohort. Compared with those patients who were excluded based on not having ECG data within 30 days of their procedure, patients with pre-operative ECGs were more likely to be older, male, and with more cardiovascular risk factors (Table 4).

TABLE 3
Characteristics and Outcomes of the Derivation Cohort

| | Total | | Training | | Validation | | Test | |
|---|---|---|---|---|---|---|---|---|
| | Cohort | | Subcohort | | Subcohort | | Subcohort | |
| No. of Patients | 45,969 | | 36,839 | | 4,549 | | 4,581 | |
| No. of Procedures | 59,975 | | 48,033 | | 6,013 | | 5,929 | |
| No. of ECGs | 112,794 | | 90,633 | | 11,217 | | 10,944 | |
| Demographic and Clinical | | | | | | | | |
| Characteristics | | | | | | | | |
| Age, years (SD) | 65.1 | (15.9) | 65.2 | (15.8) | 65.0 | (16.4) | 64.6 | (16.1) |
| Female, n (%) | 27091 | (45.2) | 21744 | (45.3) | 2706 | (45.0) | 2641 | (44.6) |
| Heart Failure, n (%) | 11000 | (18.3) | 8689 | (18.1) | 1192 | (19.8) | 1119 | (18.9) |
| Diabetes Mellitus, n (%) | 9939 | (16.6) | 7880 | (16.4) | 998 | (16.6) | 1061 | (17.9) |
| Hypertension, n (%) | 20888 | (34.8) | 16638 | (34.6) | 2187 | (36.4) | 2063 | (34.8) |
| Coronary Artery Disease, n (%) | 13019 | (21.7) | 10421 | (21.7) | 1293 | (21.5) | 1305 | (22.0) |
| Stroke, n (%) | 3392 | (5.7) | 2697 | (5.6) | 352 | (5.9) | 343 | (5.8) |
| Renal Disease, n (%) | 5683 | (9.5) | 4475 | (9.3) | 640 | (10.6) | 568 | (9.6) |
| Procedure Types | | | | | | | | |
| Cardiovascular, n (%) | 24882 | (41.5) | 19840 | (41.3) | 2595 | (43.2) | 2447 | (41.3) |
| Intraperitoneal; intrathoracic; | 10843 | (18.1) | 8679 | (18.1) | 1074 | (17.9) | 1090 | (18.4) |
| suprainguinal vascular, n (%) | | | | | | | | |
| Insulin use prior to admission, n | 3762 | (6.3) | 3022 | (6.3) | 366 | (6.1) | 374 | (6.3) |
| (%) | | | | | | | | |
| Creatinine >2 mg/dL, n (%) | 5602 | (9.3) | 4408 | (9.2) | 637 | (10.6) | 557 | (9.4) |
| RCRI >2, n (%) | 3489 | (5.8) | 2784 | (5.8) | 327 | (5.4) | 378 | (6.4) |
| Post-Operative Outcomes | | | | | | | | |
| Death during admission, n (%) | 1065 | (1.8) | 865 | (1.8) | 109 | (1.8) | 91 | (1.5) |
| Cardiovascular death, n (%) | 32 | (0.1) | 21 | (0.0) | 7 | (0.1) | 4 | (0.1) |
| Major cardiovascular events, n | 1730 | (2.9) | 1400 | (2.9) | 190 | (3.2) | 140 | (2.4) |
| (%) | | | | | | | | |
| Cardiac arrest, n (%) | 227 | (0.4) | 182 | (0.4) | 27 | (0.4) | 18 | (0.3) |
| Myocardial infarction, n (%) | 253 | (0.4) | 203 | (0.4) | 30 | (0.5) | 20 | (0.3) |
| Heart block, n (%) | 160 | (0.3) | 129 | (0.3) | 22 | (0.4) | 9 | (0.2) |
| Pulmonary edema, n (%) | 48 | (0.1) | 40 | (0.1) | 4 | (0.1) | 4 | (0.1) |

TABLE 4
Characteristics and outcomes of the derivation
cohort compared to all patients undergoing

| | All | Procedures | | | |
|---|---|---|---|---|---|
| | Procedures | with ECGs | Training | Validation | Test |
| Number of Patients | 153,465 | 45,969 | 36,839 | 4,549 | 4,581 |
| Number of Procedures | 261,328 | 59,975 | 48,033 | 6,013 | 5,929 |
| Number of ECGs | 112,794 | 112,794 | 90,633 | 11,217 | 10,944 |
| Demographics | | | | | |
| Age, years (SD) | 58.5 | 65.1 | 65.2 | 65.0 | 64.6 |
| | (19.0) | (15.9) | (15.8) | (16.4) | (16.1) |
| Female, n (%) | 142391 | 27091 | 21744 | 2706 | 2641 |
| | (54.5%) | (45.2%) | (45.3%) | (45.0%) | (44.6%) |
| Heart Failure, n (%) | 20777 | 11000 | 8689 | 1192 | 1119 |
| | (8.0%) | (18.3%) | (18.1%) | (19.8%) | (18.9%) |
| Diabetes Mellitus, n (%) | 32720 | 9939 | 7880 | 998 | 1061 |
| | (12.5%) | (16.6%) | (16.4%) | (16.6%) | (17.9%) |
| Hypertension, n (%) | 63907 | 20888 | 16638 | 2187 | 2063 |
| | (24.5%) | (34.8%) | (34.6%) | (36.4%) | (34.8%) |
| Coronary Artery Disease, | 31495 | 13019 | 10421 | 1293 | 1305 |
| n (%) | (12.1%) | (21.7%) | (21.7%) | (21.5%) | (22.0%) |
| Stroke, n (%) | 8497 | 3392 | 2697 | 352 | 343 |
| | (3.3%) | (5.7%) | (5.6%) | (5.9%) | (5.8%) |
| Renal Disease, n (%) | 14180 | 5683 | 4475 | 640 | 568 |
| | (5.4%) | (9.5%) | (9.3%) | (10.6%) | (9.6%) |
| Procedure Type | | | | | |
| Cardiovascular, n (%) | 36706 | 24882 | 19840 | 2595 | 2447 |
| | (14.0%) | (41.5%) | (41.3%) | (43.2%) | (41.3%) |
| Intraperitoneal; | 35741 | 10843 | 8679 | 1074 | 1090 |
| intrathoracic; | (13.7%) | (18.1%) | (18.1%) | (17.9%) | (18.4%) |
| suprainguinal vascular, n | | | | | |
| (%) | | | | | |
| Insulin use prior to | 12264 | 3762 | 3022 | 366 | 374 |
| admission, n (%) | (4.7%) | (6.3%) | (6.3%) | (6.1%) | (6.3%) |
| Creatinine >2 mg/dL, n | 10160 | 5602 | 4408 | 637 | 557 |
| (%) | (3.9%) | (9.3%) | (9.2%) | (10.6%) | (9.4%) |
| RCRI >2, n (%) | 6285 | 3489 | 2784 | 327 | 378 |
| | (2.4%) | (5.8%) | (5.8%) | (5.4%) | (6.4%) |
| Outcomes | | | | | |
| Death during admission, n | 1452 | 1065 | 865 | 109 | 91 |
| (%) | (0.6%) | (1.8%) | (1.8%) | (1.8%) | (1.5%) |
| Cardiovascular Mortality, | 42 | 32 | 21 | 7 | 4 |
| n (%) | (0.0%) | (0.1%) | (0.0%) | (0.1%) | (0.1%) |
| Any major cardiovascular | 2458 | 1730 | 1400 | 190 | 140 |
| complication, n (%) | (0.9%) | (2.9%) | (2.9%) | (3.2%) | (2.4%) |
| Cardiac Arrest, n (%) | 373 | 227 | 182 | 27 | 18 |
| | (0.1%) | (0.4%) | (0.4%) | (0.4%) | (0.3%) |
| Myocardial Infarction, n | 391 | 253 | 203 | 30 | 20 |
| (%) | (0.1%) | (0.4%) | (0.4%) | (0.5%) | (0.3%) |
| Heart Block n (%) | 204 | 160 | 129 | 22 | 9 |
| | (0.1%) | (0.3%) | (0.3%) | (0.4%) | (0.2%) |
| Pulmonary Edema, n (%) | 70 | 48 | 40 | 4 | 4 |
| | (0.0%) | (0.1%) | (0.1%) | (0.1%) | (0.1%) |

### Discrimination of Post-Procedural Mortality at CSMC

For the outcome of mortality, the deep learning model was developed using training, validation, and test datasets to leverage information from the single 12-lead ECG to identify post-operative death during index hospitalization. In the held-out test dataset, the deep learning model was then shown to discriminate mortality with an AUC of 0.83 (95% CI 0.79-0.87) (Table 5). By contrast, the conventional RCRI score discriminated post-operative mortality with only an AUC of 0.67 (0.61-0.72). Patients with an RCRI score of 2 or greater had an unadjusted odds ratio of 2.08 (0.77-3.50) for post-operative mortality compared to an unadjusted odds ratio of 9.17 (5.85-13.82) for patients at the same level of identified elevated risk (top 150%) by the Deep learning model algorithm. The addition of the components of the RCRI score to the Deep learning model algorithm trained only on 12-lead ECG waveforms did not significantly improve model performance (AUC 0.83; 95% CI, 0.77-0.89) in the test dataset. There were no significant differences in model performance in patient subsets by age, sex, or race (Table 6). At the pre-specified calibrated threshold of risk comparable to a RCRI score >2, the Deep learning model algorithm demonstrated a specificity of 0.87 (0.86-0.88) and sensitivity of 0.57 (0.48-0.68) for post-operative mortality. In comparison, the RCRI score >2 had slightly higher specificity of 0.94 (0.93-0.94) but much lower sensitivity of 0.12 (0.05-0.19) with similar negative and positive predictive values (Table 5).

TABLE 5
Discrimination of Post-Operative Outcomes
by the PreOpNet or RCRI Algorithms.

| | Model | | | | | | Odds |
|---|---|---|---|---|---|---|---|
| Outcome | (Cohort) | AUC | Specificity | Sensitivity | PPV | NPV | Ratio* |
| Death | PreOpNet | 0.83 | 0.87 | 0.57 | 0.06 | 0.99 | 9.17 |
| | (CSMC) | (0.79- | (0.86- | (0.48- | (0.05- | (0.99- | (5.85- |
| | | 0.87) | 0.88) | 0.68) | 0.08) | 0.99) | 13.82) |
| | PreOpNet | 0.79 | 0.86 | 0.49 | 0.03 | 1.00 | 6.20 |
| | (CUMC) | (0.75- | (0.80- | (0.48- | (0.02- | (0.99- | (3.87- |
| | | 0.83) | 0.92) | 0.50) | 0.03) | 1.00) | 10.41) |
| | PreOpNet | 0.75 | 0.93 | 0.32 | 0.02 | 1.00 | 5.88 |
| | (SHC) | (0.74- | (0.91- | (0.32- | (0.02- | (1.00- | (5.00- |
| | | 0.76) | 0.94) | 0.32) | 0.02) | 1.00) | 7.00) |
| | RCRI | 0.67 | 0.94 | 0.12 | 0.03 | 0.99 | 2.08 |
| | | (0.61- | (0.93- | (0.05- | (0.01- | (0.98- | (0.77- |
| | | 0.72) | 0.94) | 0.19) | 0.05) | 0.99) | 3.50) |
| MACE | PreOpNet | 0.77 | 0.88 | 0.41 | 0.08 | 0.98 | 5.38 |
| | (CSMC) | (0.73- | (0.88- | (0.33- | (0.06- | (0.98- | (3.75- |
| | | 0.80) | 0.89) | 0.49) | 0.10) | 0.99) | 7.49) |
| | RCRI | 0.63 | 0.94 | 0.10 | 0.04 | 0.98 | 1.67 |
| | | (0.59- | (0.93- | (0.05- | (0.02- | (0.97- | (0.77- |
| | | 0.68) | 0.94) | 0.15) | 0.06) | 0.98) | 2.68) |

AUC = Area under the curve. MACE = Major adverse cardiovascular events. RCRI = Revised Cardiovascular Risk Index. CSMC denotes results for the internal held-out test dataset from the derivation cohort, and SHC and CUMC denote results for the external validation cohorts.
*Unadjusted odds ratios for the outcome of interest (death or MACE) were calculated for patients in the ≥85^{th} percentile of risk compared to those in the <85^{th} percentile of risk, as determined by either PreOpNet or the RCRI (corresponding go an RCRI score ≥2).

TABLE 6
Performance of PreOpNet stratified by demographic groups

| Outcome | Demographic | AUC |
|---|---|---|
| MACE | Female | 0.80 (0.74-0.87) |
| | Male | 0.74 (0.69-0.79) |
| | Black | 0.72 (0.57-0.86) |
| | Caucasian | 0.76 (0.71-0.81) |
| | Other Ethnicity | 0.82 (0.75-0.89) |
| | Under 55 years old | 0.79 (0.68-0.89) |
| | 55-75 years old | 0.77 (0.72-0.82) |
| | Older than 75 years old | 0.74 (0.66-0.81) |
| Death | Female | 0.87 (0.81-0.94) |
| | Male | 0.80 (0.75-0.85) |
| | Black | 0.84 (0.77-0.91) |
| | Caucasian | 0.83 (0.77-0.88) |
| | Other Ethnicity | 0.83 (0.74-0.92) |
| | Under 55 years old | 0.84 (0.75-0.92) |
| | 55-75 years old | 0.87 (0.83-0.91) |
| | Older than 75 years old | 0.75 (0.64-0.85) |

### Discrimination of Post-Procedural Mortality in Important Clinical Scenarios

The deep learning model performed well both in patients undergoing major surgical procedures in the operating room as well as patients undergoing procedures in the catheterization laboratory or endoscopy suite. For patients with surgeries in the operating room, Deep learning model discriminated post-operative mortality with an AUC of 0.84 (0.76-0.92), compared to an AUC of 0.70 (0.61-0.78) for the RCRI score. For patients with procedures in the catheterization lab or endoscopy suite, the deep learning model discriminated post-operative mortality with an AUC of 0.83 (0.78-0.98), compared to an AUC of 0.66 (0.60-0.72) for the RCRI score. The deep learning model performed similarly in discriminating mortality in patients undergoing cardiovascular surgery with an AUC of 0.85 (0.77-0.92) and patients undergoing non-cardiac surgery with an AUC of 0.83 (0.79-0.88). In patients undergoing cardiac surgery, the RCRI score discriminated post-operative mortality with an AUC of 0.62 (0.52-0.72), and in patients undergoing non-cardiac surgery, the RCRI score discriminated post-operative mortality with an AUC of 0.70 (0.63-0.77).

Given that ECGs are often not obtained in low-risk patients undergoing low-risk procedures, a secondary analysis was performed in patients most likely to be considered at least moderate-risk-patients either with known cardiovascular disease or those undergoing elective intermediate-risk or high-risk surgery. Without additional subset-specific fine-tuning, the deep learning model discriminated post-operative mortality in this subset with an AUC of 0.80 (0.71-0.88). In clinical practice, pre-operative risk assessment most commonly occurs in the elective procedural setting. Thus, secondary analyses were performed limited to those patients in the CSMC test cohort who were undergoing elective procedures (3,691 patients contributing 5,165 ECGs). In this setting, the deep learning model algorithm discriminated post-operative mortality with an AUC of 0.80 (0.67-0.92).

### Discrimination of Post-Procedural Mortality in External Validation Cohorts

To assess external validity of the Deep learning model algorithm, the analyses above were repeated on cohorts from external health system cohorts using the algorithm without any additional tuning. The external test evaluation cohorts included 101,375 patients in the Stanford Healthcare (SHC) system contributing 162,540 ECGs and 9,028 patients in the Columbia University Medical Center (CUMC) system contributing 9,028 ECGs. In the SHC cohort, the post-operative mortality rate was 1.3% and the deep learning model discriminated this outcome with an AUC of 0.75 (0.74-0.76). In the CUMC cohort, the post-operative mortality rate was 1.6% and the algorithm discriminated this outcome with an AUC of 0.79 (0.75-0.83) (Table 5). The Deep learning model algorithm pre-specified high risk group (>15%) had an unadjusted odds ratio of 5.88 (5.00-7.00) in SHC and 6.20 (3.87-10.41) in CUMC for mortality. Results from analyses of specificity, sensitivity, and positive and negative predictive value were similar in the external validation cohorts when compared with results observed in the CSMC cohort (Table 5).

### Deep Learning Model Discrimination of Major Cardiovascular Events at CSMC

Secondary analyses using the deep learning model to evaluate both post-operative mortality and non-fatal MACE were performed. For this secondary outcome, the deep learning model algorithm discriminated events in the held-out test dataset with an AUC of 0.77 (0.73-0.80) whereas the RCRI score had an AUC of 0.63 (0.59-0.68). Patients with an RCRI score of 2 or greater had an unadjusted odds ratio of 1.67 (0.77-2.68) for mortality or MACE when compared with those with an RCRI score <2. By contrast, patients identified by the Deep learning model algorithm to be high risk had an unadjusted odds ratio 5.38 (3.75-7.49) for post-operative mortality or MACE (Table 5). The Deep learning model algorithm demonstrated a specificity of 0.88 (0.88-0.89) and sensitivity of 0.41 (0.33-0.49) while the RCRI score again had higher specificity at 0.94 (0.93-0.94) but much lower sensitivity at 0.10 (0.05-0.15) with similar negative and positive predictive values (Table 5). FIG. 10A shows receiver operating curves depicting performance of the deep learning model at Cedars-Sinai Medical Center (CSMC, plot 1002), Stanford Healthcare (SHC, plot 1004), and Columbia University Medical Center (CUMC, plot 1006), and Revised Cardiac Risk Index (plot 1008) in discriminating post-operative mortality. Further FIG. 10B shows receiver operating curves for CSMC (plot 1052) and RCRI (plot 1054) in discriminating major adverse cardiovascular events.

### Clinical Risk Assessment and Reclassification

The ability of the deep learning model algorithm to reclassify risk in the hold-out test dataset was evaluated. When compared with the RCRI score, application of the deep learning model led to significant improvement in the continuous net reclassification index (NRI 0.53; 95% CI, 0.38 to 0.68). In categorical analyses using the pre-specified threshold of risk (top 15th percentile Deep learning model prediction), 981 (82.4%) of 1190 patients originally classified as high-risk for MACE by the RCRI score (RCRI >2) were identified as low-risk by deep learning model (Table 7). Of these high-to-low reclassified risk patients, 33 (3.4%) patients experienced MACE. By contrast, of the 4,739 patients classified as low-risk by RCRI (RCRI <2), the Deep learning model algorithm reclassified 327 (6.9%) to be high risk; of these patients, 26 (8.0%) patients experienced MACE. Despite a fair amount of reclassification, the categorical NRI at this cut-point was not significant (NRI 0.06; 95% CI −0.04 to 0.18) for MACE.

TABLE 7
Net Reclassification Table for Major Averse Cardiovascular Events (MACE).

| | | PreOpNet Risk Classification | | | |
|---|---|---|---|---|---|
| | | 0-8% Risk of | Greater than 8% | | No. (%) |
| | | MACE | Risk of MACE | Total | Reclassified |
| RCRI | Low Risk (Score <2) | | | | |
| Score | Number of participants | 4412 | 327 | 4739 | 327 (6.9%) |
| | Percent of participants | 74.4 | 5.5 | | |
| | MACE | 57 (1.3%) | 26 (8.0%) | | |
| | High Risk (Score >=2) | | | | |
| | Number of participants | 981 | 209 | 1190 | 981 (82.4%) |
| | Percent of participants | 16.6 | 3.5 | | |
| | MACE | 33 (3.4%) | 24 (11.5%) | | |
| | Total | 5393 | 536 | 5929 | 1308 (22.1%) |

RCRI = Revised Cardiac Risk Index

Comparison of Deep Learning Model Performance with Alternative ECG Assessment Models

The AUC for post-operative mortality and MACE was greater for Deep learning model as compared with traditional ECG measures (e.g. heart rate, axis, and intervals), physician ECG interpretations, or clinical variables in the hold-out test dataset (Table 8). An XGB model on RCRI variables performed less well than Deep learning model and performed similarly to the RCRI score in discriminating MACE with an AUC of 0.62 (0.59-0.65) and post-operative mortality with an AUC of 0.70 (0.66-0.74). To clarify how functionality of the Deep learning model algorithm might be interpreted in clinical context, Local Interpretable Model-agnostic Explanations (LIME)^{9,10} was applied to the data. The LIME sensitivity analyses highlighted features of the QRS complexes as the most relevant to model decision making; in addition, precordial premature contractions and intraventricular block on the precordial leads were also frequently highlighted as part of this secondary analysis (FIGS. 11A and 11B).

TABLE 8
PreOpNet model performance compared to non-deep learning based risk
prediction models at CSMC. The alternative models with inputs of
1) the combination of ECG measurements, physician ECG interpretations,
and clinical variables, 2) all ECG measurements obtained from MUSE
including heart rate, axis, and intervals, 3) physician ECG interpretations,
and 4) clinical variables from the RCRI score.

| Outcome | Model | AUC |
|---|---|---|
| Mortality | PreOpNet | 0.83 (0.78-0.88) |
| | ECG Measurements, Interpretation, RCRI | 0.64 (0.63-0.65) |
| | clinical variables | |
| | ECG Measurements (Intervals, Axis, HR, | 0.66 (0.65-0.67) |
| | etc) | |
| | Physician Overread Interpretation of ECGs | 0.64 (0.63-0.65) |
| | Clinical Variables from RCRI Score | 0.65 (0.55-0.74) |
| MACE | PreOpNet | 0.77 (0.74-0.80) |
| | ECG Measurements, Interpretation, RCRI | 0.74 (0.73-0.75) |
| | clinical variables | |
| | ECG Measurements (Intervals, Axis, HR, | 0.60 (0.59-0.61) |
| | etc) | |
| | Physician Overread Interpretation of ECGs | 0.63 (0.62-0.63) |
| | Clinical Variables from RCRI Score | 0.61 (0.51-0.70) |

TABLE 9
Performance and computational complexity
of deep learning models.

| | FLOPs* | Training Time | |
|---|---|---|---|
| PreOpNet | 57,913,936 | 82 | minutes |
| Attia et al. | 95,773,024 | 103 | minutes |
| Kwon et al. | 199,355,858 | 194 | minutes |
| Goto et al. | 3,155,739,318 | 1320 | minutes |

FLOPs = Floating point operations

TABLE 10
PreOpNet model performance at various thresholds of risk

| | | Odds Ratio | | | | |
|---|---|---|---|---|---|---|
| Outcome | Threshold | For Event | Specificity | Sensitivity | PPV | NPV |
| Death | Max | 9.48 (5.99- | 0.82 (0.81- | 0.66 (0.56- | 0.06 (0.04- | 0.99 (0.99- |
| | Youden's | 14.91) | 0.83) | 0.76) | 0.07) | 1.00) |
| | index | | | | | |
| | Top 7% of | 8.83 (5.57- | 0.90 (0.89- | 0.48 (0.38- | 0.07 (0.05- | 0.99 (0.99- |
| | Risk | 13.20) | 0.91) | 0.59) | 0.09) | 0.99) |
| | Top 15% of | 9.17 (5.85- | 0.87 (0.86- | 0.57 (0.48- | 0.06 (0.05- | 0.99 (0.99- |
| | Risk | 13.82) | 0.88) | 0.68) | 0.08) | 0.99) |
| | Top 50% of | 10.47 (5.34- | 0.53 (0.52- | 0.89 (0.82- | 0.03 (0.02- | 1.00 (0.99- |
| | Risk | 24.47) | 0.54) | 0.96) | 0.03) | 1.00) |
| Death or | Max | 5.31 (3.41- | 0.54 (0.52- | 0.81 (0.75- | 0.04 (0.03- | 0.99 (0.99- |
| non-fatal | Youden's | 8.41) | 0.55) | 0.88) | 0.05) | 0.99) |
| MACE | index | | | | | |
| | Top 7% of | 6.15 (4.13- | 0.92 (0.91- | 0.36 (0.28- | 0.09 (0.07- | 0.98 (0.98- |
| | Risk | 8.58) | 0.92) | 0.44) | 0.12) | 0.99) |
| | Top 15% of | 5.38 (3.75- | 0.88 (0.88- | 0.41 (0.33- | 0.08 (0.06- | 0.98 (0.98- |
| | Risk | 7.49) | 0.89) | 0.49) | 0.10) | 0.99) |
| | Top 50% of | 5.30 (3.40- | 0.54 (0.52- | 0.81 (0.75- | 0.04 (0.03- | 0.99 (0.99- |
| | Risk | 8.39) | 0.55) | 0.88) | 0.05) | 0.99) |

### Technical Application

To facilitate the potential pragmatic application of the deep learning model algorithm as a decision support tool in the clinical setting, the deep learning model developed using deep learning algorithms was optimized to run on a standard-build clinical workstation including a computer without graphical processing units (GPUs) and also developed a web application to facilitate access by clinicians (FIG. 13) In a series of repeated runtime experiments, the deep learning model application accessed on a standard clinical workstation (Windows 10 64-bit OS, 3.8 GHz processor) was able to intake image data from 50 de novo ECGs and output post-operative risk estimates within 0.032±0.004 seconds per ECG for the local software installation and 0.041±0.006 seconds per ECG for the web application accessed by a mobile phone.

### Discussion

In a large cohort of patients undergoing inpatient procedures, a deep learning algorithm utilizing the waveforms of a single pre-operative 12-lead ECG identified risk for post-operative death for cardiac surgeries, non-cardiac surgeries, and catheterization lab interventions. Compared with a widely used standard pre-operative risk assessment tool, the deep learning model was able to more effectively identify high-risk patients who went on to experience postoperative mortality. Further, the accuracy of deep learning model for discriminating post-operative mortality was re-affirmed in two external healthcare system cohorts with diverse patient populations. Additionally, the deep learning model was shown to identify high-risk patients who experience MACE in the CSMC patient population. The deep learning model is the first deep learning architecture designed to aid clinicians in discriminating post-operative outcomes. The deep learning model improves efficacy as well as efficiency of the pre-operative risk assessment.

Conventional approaches have evolved over the past three decades to include clinical risk factors as well as both biomarkers and imaging diagnostics. However, it has proved difficult to achieve greater than modest accuracy in the prediction of post-operative outcomes. At the same time, concerns surrounding the costs and burden of potentially unnecessary pre-operative testing have continued to mount. Further, the inventors have recognized that a persistent challenge in achieving more accurate prediction of post-operative outcomes is the marked heterogeneity of patients at risk, which amplifies the potential importance of unmeasured variables. To address the above-mentioned challenges, the deep learning model development focused on deriving new prognostic information from the pre-operative 12-lead ECG given its wide accessibility as a diagnostic that is already frequently ordered in clinical practice.

Over the last several years, deep learning approaches for ECG have been used to improve detection of cardiovascular traits (e.g. atrial fibrillation, hypertrophic cardiomyopathy, cardiac amyloidosis, and ventricular dysfunction) and also to identify non-cardiovascular specific traits such as liver disease, anemia, age, and long-term mortality. However, none the previous approaches address risk of post-operative or post-procedural or post-treatment outcomes. The previous models for ECG analysis lack actionability and cannot be used to determine what is the level of mortality risk or MACE risk pre-operatively for a patient within a future post-operative period. Further, other non-deep learning models rely on post-operative biomarkers, which limits the opportunity to stratify risk before operation.

The deep learning method discussed herein address the above mentioned issues and uses readily available ECG waveform data (e.g., a single 10 second ECG time series data) from and is developed using large diverse real-world patient cohorts. As shown above, the resulting deep learning model demonstrated ability not only to discriminate post-operative mortality, but to do so while outperforming a conventional clinical risk score and human-interpretable features from the ECG. The deep learning method described herein leverages latent features from ECG to improve diagnosis or prognostication, and the results demonstrate the potential value of the deep learning model algorithm to augment clinical decision making for pre-operative risk assessment.

For deep learning algorithms to be readily integrated in the clinical practice setting, implementation needs to pose little to no additional burden to existing clinical workflows. Although not the primary focus of the current study, we took several steps towards assessing the potential application of Deep learning model in practice. Recognizing that many image-based, including ECG focused algorithms, tend to be computationally resource intensive, the deep learning model can be run on a standard-build clinical workstation including a computer that does not include graphical processing units (GPUs) as part of its configuration. The runtime from local and web-based installations was assessed and found to have an acceptable real-time implementation duration of <1 second.

The deep learning model discussed herein offers several strengths including the ability to leverage internal training, validation, and test datasets within a large derivation cohort of patients undergoing inpatient procedures over a decade of time. The algorithm was also able to be externally validated for post-procedural mortality across three large, diverse medical centers.

In summary, the results shown above demonstrate how a novel deep learning algorithm, applied to a single pre-operative ECG, can improve discrimination of post-operative adverse outcomes while running efficiently on a standard clinical workstation. Further, the deep learning method for post-operative risk stratification provided herein is efficiently and effectively integrated with existing clinical workflows, and improves patient outcomes.

### Computer & Hardware Implementation of Disclosure

It should initially be understood that the disclosure herein may be implemented with any type of hardware and/or software, and may be a pre-programmed general purpose computing device. For example, the system may be implemented using a server, a personal computer, a portable computer, a thin client, a wearable device, a digital stethoscope, or any suitable device or devices. The disclosure and/or components thereof may be a single device at a single location, or multiple devices at a single, or multiple, locations that are connected together using any appropriate communication protocols over any communication medium such as electric cable, fiber optic cable, or in a wireless manner.

It should also be noted that the disclosure is illustrated and discussed herein as having a plurality of modules which perform particular functions. It should be understood that these modules are merely schematically illustrated based on their function for clarity purposes only, and do not necessary represent specific hardware or software. In this regard, these modules may be hardware and/or software implemented to substantially perform the particular functions discussed. Moreover, the modules may be combined together within the disclosure, or divided into additional modules based on the particular function desired. Thus, the disclosure should not be construed to limit the present invention, but merely be understood to illustrate one example implementation thereof.

In some implementations, the computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Implementations of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks), and any wireless networks.

Implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively or in addition, the program instructions can be encoded on an artificially-generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium can also be, or be included in, one or more separate physical components or media (e.g., multiple CDs, disks, flash memory, or other storage devices).

The operations described in this specification can be implemented as operations performed by a “data processing apparatus” on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, multi-core processors, GPUs, AI-accelerators, In-memory computing architectures or combinations, of the foregoing. The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures, and deep learning and artificial intelligence computing infrastructure.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit). Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, flash memory or optical disks. However, a computer need not have such devices. Moreover, a computer can be embedded in another device, e.g., a mobile telephone, a personal digital assistant (PDA), smart watch, smart glasses, patch, wearable devices, a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

### CONCLUSION

The various methods and techniques described above provide a number of ways to carry out the invention. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Certain embodiments of this application are described herein. Variations on those embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

Particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

1. A system for performing pre-operative risk assessment, the system comprising:
at least one memory storing a trained neural network model and executable instructions;
at least one processor communicably coupled to the at least one memory and when executing the instructions cause the processor to:
receive electrocardiogram (ECG) waveform data of a patient, the ECG waveform data acquired via an ECG sensor system and acquired within a threshold period before a desired procedure;
input the ECG waveform data in to the trained neural network model;
obtain as output from the trained neural network model, one or more post-operative risk metrics; and
display, via a display portion of a user interface coupled to the at least one processor, the one or more post-operative risk metrics.

2. The system of claim 1, wherein the trained neural network model is trained using a set of ECG waveform data, the set of ECG waveform data acquired from a patient population and within the threshold period before corresponding medical procedures and labelled with corresponding post-operative outcomes for mortality within a post-operative period.

3. The system of claim 1, wherein the trained neural network model is trained using a second set of ECG waveform data, the second set of ECG waveform data acquired from a patient population within the threshold period before corresponding medical procedures and labelled with corresponding post-operative outcomes for one or more non-fatal major adverse cardiac events within a post-operative period.

4. The system of claim 1, wherein the one or more post-operative risk metric includes one or more of a post-operative mortality risk score and a post-operative MACE risk score; and wherein the one or more post-operative major adverse cardiac events includes one or more of a cardiac arrest, a myocardial infarction, a heart block, and a pulmonary edema.

5. The system of claim 1, wherein the trained neural network comprises at least one atrous convolutional layer.

6. The system of claim 1, wherein the desired procedure is selected from the group consisting of a cardiac surgery, a non-cardiac surgery, an endoscopic procedure, a catheterization lab procedure, a non-surgical procedure, a radiation therapy, an immunotherapy, a chemotherapy, and any combination thereof.

7. The system of claim 5, wherein the at least one atrous convolutional layer is an initial layer of the trained neural network model and receives the normalized ECG waveform data as input.

8. The system of claim 7, wherein the trained neural network model comprises a plurality of inverted residual layers downstream of the at least one atrous convolutional layer, each of the plurality of the inverted residual layers comprising one or more bottleneck modules, each of the one or more bottleneck modules configured to output a second number of channels greater than a first number of channels input into each of the one or more bottleneck modules.

9. The system of claim 8, the trained neural network model further comprises a non-atrous convolutional layer receiving input from a last inverted residual layer, and a fully connected layer receiving an average pooled input from the non-atrous convolutional layer; and wherein the non-atrous convolutional layer applies a one dimensional non-atrous convolutional operation on its input.

10. The system of claim 1, wherein the at least one memory stores further instructions that when executed cause the processor to:
apply a model interpretable function to the ECG waveform data one or more post-operative risk metrics output by the trained neural network model;
determine one or more relevant ECG features contributing to the one or more post-operative risk metrics based on the model interpretable function; and
indicate the one or more relevant ECG features via the user interface.

11. The system of claim 10, wherein the model interpretable function is configured according to a specified Local Interpretable Model-Agnostic Explanations (LIME) model for the ECG waveform data.

12. The system of claim 1, wherein the at least one memory stores further instructions that when executed cause the processor to:
determine whether the patient is recommended for the intended procedure based on the one or more post-operative risk metrics; and
output the recommendation via the user interface.

13-22. (canceled)

23. A method for performing pre-operative risk assessment for a patient, the method comprising:
receiving, at a processor, an electrocardiogram (ECG) waveform data acquired from an ECG sensor system, the ECG waveform data acquired within a pre-operative period prior to a medical procedure or treatment; and
processing, via the processor, the ECG waveform data using a first trained neural network model to generate a first output including a risk of post-operative mortality;
processing, via the processor, the ECG waveform data using a second trained neural network model to generate a second output including a risk of one or more non-fatal major adverse cardiac events; and
displaying, via a display coupled to the processor, the first output and the second output;
wherein the first trained neural network model is trained using a first training dataset comprising a plurality of ECG waveforms, each of the plurality of ECG waveforms acquired within the pre-operative period before a respective procedure or treatment, and labelled with a corresponding post-operative mortality outcome; and
wherein the second trained neural network model is trained with a second training dataset comprising a plurality of ECG waveforms acquired within the pre-operative period before a respective procedure or treatment, and labelled with a corresponding post-operative major adverse cardiac event outcome.

24. The method of claim 23, further comprising determining, via the processor, one or more first relevant ECG features of the ECG waveform data based on a model interpretability function, the one or more first relevant ECG features contributing to the first output; and determining, via the processor, one or more second relevant ECG features of the ECG waveform data based on the model interpretability function, the one or more second relevant ECG features contributing to the second output.

25. The method of claim 23, wherein each of the first and the second neural network models comprise an atrous convolutional layer receiving the ECG waveform data as input.

26. The method of claim 23, wherein the one or more post-operative major adverse cardiac events include one or more of a cardiac arrest, a myocardial infarction, a heart block, and a pulmonary edema.

27. The method of claim 23, wherein the ECG waveform data is a time series data.

28. The method of claim 23, wherein the medical procedure or treatment is selected from the group consisting of a cardiac surgery, a non-cardiac surgery, an endoscopic procedure, a catheterization lab procedure, a non-surgical procedure, a radiation therapy, an immunotherapy, a chemotherapy, and any combination thereof.

29. The method of claim 23, wherein the first and the second neural network models each have an area under the curve (AUC) of 0.75 or greater than 0.75.

30. The method of claim 23, further comprising: determining whether the patient is recommended to undergo the medical procedure or treatment based on one or more of the first and the second outputs, and displaying the recommendation on the display.
